(19) United States
(12) Patent Application Publication
(10) Pub. No.: US 2022/0195494 A1
(43) Pub. Date: Jun. 23, 2022

(54) STABILIZATION AND PRESERVATION OF IN VITRO TRANSCRIPTION REACTIONS

(71) Applicants: Northwestern University, Evanston, IL (US); Massachusetts Institute of Technology, Cambridge, MA (US); President and Fellows of Harvard College, Cambridge, MA (US)

(72) Inventors: Khalid K. Alam, Evanston, IL (US); Matthew S. Verosloff, Evanston, IL (US); Jaeyoung K. Jung, Evanston, IL (US); Julius B. Lucks, Evanston, IL (US); James J. Collins, Newton, MA (US); Phillip R. Clauer, Evanston, IL (US); Jeong Wook Lee, Evanston, IL (US); Peter Q. Nguyen, Evanston, IL (US)

(21) Appl. No.: 17/606,441

(22) PCT Filed: Apr. 27, 2020
(86) PCT No.: PCT/US2020/030112

§ 371 Date: Oct. 25, 2021

Related U.S. Application Data
(60) Provisional application No. 62/838,852, filed on Apr. 25, 2019.

(51) Int. Cl. C12Q 1/6806

(52) U.S. Cl. CPC C12Q 1/6806

(57) ABSTRACT

The disclosed subject matter relates to compositions, systems, kits, and methods that typically comprise and/or utilize a lyophilized composition comprising components for performing in vitro transcription prepared by lyophilizing an aqueous composition that includes: (i) the one or more components for performing in vitro transcription; and (ii) a non-reducing polysaccharide at a concentration of at least about 40 mM and/or a sugar alcohol at a concentration of at least about 40 mM. In some embodiments, the lyophilized composition is prepared by lyophilizing an aqueous composition that comprises no more than 5% (v/v) glycerol.

### CROSS-REFERENCED TO RELATED PATENT APPLICATIONS

The present application claims the benefit of priority under 35 U.S.C. § 119(e) to U.S. Provisional Application No. 62/838,852, filed on Apr. 25, 2019, the content of which is incorporated herein by reference in its entirety.

### BACKGROUND

The present invention is related to compositions, systems, kits, and methods for performing transcription in vitro. The compositions, systems, kits, and methods utilize components and mixtures that are stabilized and preserved through lyophilization.

In vitro transcription (IVT) is the process of synthesizing a polymer of nucleic acids (typically RNA) from a transcription template (typically DNA). IVT reactions typically consist of an RNA polymerase, a DNA transcription template, nucleotide triphosphates (NTP), and a buffered reaction mixture with cofactors. IVT is used in educational settings, for research and development purposes, for medical, agricultural and industrial applications, and more recently, to create low-cost and rapid biosensors.

However, there is currently no known way to stabilize an IVT reaction mixture for distribution in the absence of cold-chain. This is despite a number of methods which exist for freeze-drying other biochemical reactions. Addressing this need would allow for low-cost, on-site, and on-demand production of RNA for the aforementioned purposes by simply rehydrating the reaction. Here we describe how to successfully lyophilize (freeze-dry) IVT reactions and show how our method/formulation allows for the preservation of a biosensor based on IVT. We show that several common lyoprotectants offer little or no lyoprotection when applied to an IVT, identify those that do offer meaningful lyoprotection, and describe an optimal formulation.

### SUMMARY

The disclosed subject matter relates to compositions, systems, kits, and methods that typically comprise and/or utilize a lyophilized composition comprising components for performing in vitro transcription prepared by lyophilizing an aqueous composition that includes: (i) the one or more components for performing in vitro transcription; and (ii) a non-reducing polysaccharide at a concentration of at least about 40 mM and/or a sugar alcohol at a concentration of at least about 40 mM. In some embodiments, the lyophilized composition is prepared by lyophilizing an aqueous composition that comprises no more than 5% glycerol (v/v).

### BRIEF DESCRIPTION OF THE FIGURES

FIG. 1. Effect of freeze-drying (FD) components on in vitro transcription in a reaction mixture. Fresh: non-freeze-dried reaction mixture. DFHBI-T1 FDed: The dye 3′5′-difluoro-4-hydroxybenzylidene imidazolinone (“DFHBI-T1 Fded”) was freeze-dried and added to a fresh reaction mixture. T7 RNA pol FDed: T7 RNA polymerase was freeze-dried and was added to a fresh reaction mixture. FD: The entire reaction mixture including DFHBI-T1 dye and T7 RNA polymerase was freeze-dried.

FIG. 2. Effect of polyethylene glycol (8000) at various concentrations independently and in combination with bovine serum albumin (BSA) at various concentrations on in vitro transcription after freeze-drying a reaction mixture. (See FIGS. 2A and 2B).

FIG. 3. Effect of milk powder on in vitro transcription after freeze-drying a reaction mixture.

FIG. 4. Effect of various sugars on in vitro transcription after freeze-drying a reaction mixture: glucose (Glu), fructose (Fm), L-arabinose (L-ara), D-arabinose (D-ara).

FIG. 5. Effect of various sugars and sorbitol on in vitro transcription after freeze-drying a reaction mixture: cellobiose (Cel), lactose (Lac), maltose (Mal), and sorbitol (Sor).

FIG. 6. Effect of D-mannitol on in vitro transcription after freeze-drying a reaction mixture.

FIG. 7. Effect of sucrose and trehalose at various concentrations on in vitro transcription after freeze-drying a reaction mixture.

FIG. 8. Effect of sucrose at various concentrations on in vitro transcription after freeze-drying a reaction mixture. FIG. 8A: 20 mM, 40 mM, 80 mM, 120 mM, 160 mM, and 200 mM. FIG. 8B: 200 mM, 250 mM, 300 mM, 350 mM, and 400 mM.

FIG. 9. Effect of non-reducing trisaccharides maltotriose and raffinose at various concentrations on in vitro transcription after freeze-drying a reaction mixture.

FIG. 10. Effect of high concentrations of sucrose and trehalose on in vitro transcription in a fresh reaction mixture (i.e., not a freeze-dried reaction mixture).

FIG. 11. Effect of glycerol on in vitro transcription after freeze-drying a reaction mixture.

FIG. 12. Effect of sucrose in combination with glycine on in vitro transcription after freeze-drying a reaction mixture.

FIG. 13. Effect of sucrose in combination with D-mannitol on in vitro transcription after freeze-drying a reaction mixture.

FIG. 14. Rehydration of inducible in vitro transcription reaction after freeze-drying with sucrose. Ctc: chlortetracycline; CtcS: transcription repressor used to prevent transcription unless induced by chlortetracycline (Ctc).

FIG. 15. Effect of sucrose and D-mannitol on inducible in vitro transcription after freeze-drying a reaction mixture containing the inducible copper repressor CsoR and rehydration with and without Cu²⁺.

FIG. 16. Rehydration of lyophilized CsoR-regulated (A) or CadC-regulated (B) in vitro transcription reactions using real-world water samples spiked with Cu²⁺ (A) or Pb²⁺ (B). CsoR: inducible copper sensor; CadC: inducible lead sensor. Sucrose and D-mannitol were used as lyo-protectants.

FIG. 17. Rehydration of lyophilized regulated in vitro transcription reactions using real-world municipal water sources spiked with copper (A) or zinc (B). CsoR: inducible copper sensor; SmtB: inducible zinc sensor: NIMPLY: copper and not-zinc logic gate. Sucrose and D-mannitol were used as lyo-protectants.

FIG. 18. Rehydration of lyophilized regulated in vitro transcription reactions using environmental water source spiked with copper. CsoR: inducible copper sensor; SmtB: inducible zinc sensor: NIMPLY: copper and not-zinc logic gate. Sucrose and D-mannitol were used as lyo-protectants.

FIG. 19. Rehydration of lyophilized copper sensor using environmental water sources from Chile known to be contaminated with copper.

FIG. 20. Decreased performance versus time for rehydration inducible in vitro transcription reaction mixtures using aTc as a transcription inducer and TetR as a transcription regulator. Reaction mixtures were stored with desiccant and without protection from light.

FIG. 21. Effect of purging with inert gas and light protection on in vitro transcription versus time in storage. (A) Purging and storage process. (B) Induced transcription in the presence of aTc.

FIG. 22. Detection of zinc and copper using rehydrated inducible in vitro transcription reaction mixtures in field samples. (A) Preparation and shipment of lyophilized inducible in vitro transcription reaction mixtures to the site of interest for rehydration with field samples. (B) Experimental set-up. CsoR: inducible copper sensor; SmtB: inducible zinc sensor: NIMPLY: copper and not-zinc logic gate. (C)(D) Detection of zinc in field sample (C) Municipal Water 1; (D) Municipal Water 2. (E)(F) Detection of zinc and copper in field sample (E) Municipal Water 3; (F) Municipal Water 4.

### DETAILED DESCRIPTION

The present invention is described herein using several definitions, as set forth below and throughout the application.

Unless otherwise specified or indicated by context, the terms “a”, “an”, and “the” mean “one or more.” For example, “a composition,” “a system,” “a kit,” “a method,” “a protein,” “a vector,” “a domain,” “a binding site,” “an RNA,” “a non-reducing disaccharide,” and “a sugar alcohol,” should be interpreted to mean “one or more compositions,” “one or more systems,” “one or more kits,” “one or more methods,” “one or more proteins,” “one or more vectors,” “one or more domains,” “one or more binding sites,” “one or more RNAs,” “one or more non-reducing disaccharides,” and “one or more non-reducing sugar alcohols,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “regulation” and “modulation” may be utilized interchangeably and may include “promotion” and “induction.” For example, a transcription factor that regulates or modulates expression of a target gene may promote and/or induce expression of the target gene. In addition, the terms “regulation” and “modulation” may be utilized interchangeably and may include “inhibition” and “reduction.” For example, a transcription factor that regulates or modulates expression of a target gene may inhibit and/or reduce expression of the target gene.

As used herein, the term “sample” may include “biological samples” and “non-biological samples.” Biological samples may include samples obtained from a human or non-human subject. Biological samples may include but are not limited to, blood samples and blood product samples (e.g., serum or plasma), urine samples, saliva samples, fecal samples, perspiration samples, and tissue samples. Non-biological samples may include but are not limited to aqueous samples (e.g., watershed samples) and surface swab samples.

“Non-reducing polysaccharides” are known in the art. As would be understood in the art, “non-reducing polysaccharides” include polysaccharides (e.g., disaccharides and larger polysaccharides) which lack a free aldehyde or a free ketone group. As would be understood in the art, “non-reducing polysaccharides” include polysaccharides (e.g., disaccharides and larger polysaccharides) which do not act as reducing agents. For example, non-reducing polysaccharides may have glycosidic bonds between their anomeric carbons and thus cannot convert to an open-chain form with a free aldehyde group (i.e., they are fixed in the cyclic form). A free aldehyde group can act as a reducing agent in tests such as the Tollens' test or Benedict's test. As such, reducing polysaccharides can be detected in tests such as the Tollens' test or Benedict's test, while non-reducing polysaccharides are not detected in tests such as the Tollens' test or Benedict's test.

As used herein, the term “lyophilizing” refer a process wherein a thing is preserved by freezing the thing very quickly and then by subjecting the frozen thing to a vacuum which removes ice from the frozen thing. In some instance, “lyophilization” may be alternatively referred to as “freeze-drying.”

Polynucleotides and Uses Thereof

The compositions disclosed herein may include polynucleotides and/or may be utilized to synthesize polynucleotides (e.g. via in vitro RNA transcription). The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may comprise polymers of ribonucleotides containing D-ribose otherwise referred to as NTP's polymers of deoxyribonucleotides containing 2-deoxy-D-ribose otherwise referred to as dNTP's, and to polymers of any other type of nucleotide that is an N glycoside of a purine or pyrimidine base. The compositions disclosed herein may include NTP's, dNTP's, and/or any other type of nucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “promoter” refers to a cis-acting DNA sequence that directs RNA polymerase and other trans-acting transcription factors to initiate RNA transcription from the DNA template that includes the cis-acting DNA sequence.

As used herein, “expression template” refers to a nucleic acid that serves as substrate for transcribing at least one RNA. Expression templates include nucleic acids composed of DNA or RNA. Suitable sources of DNA for use in a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Peptides, Polypeptides, and Proteins

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” or “polypeptide” or “peptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) or non-naturally occurring amino acids. The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation), hydroxylation, iodination (e.g., of thyroid hormones), and phosphorylation (e.g., the addition of a phosphate group, usually to serine, tyrosine, threonine or histidine).

Polymerases

The compositions disclosed herein may include polymerases. As used herein, a “polymerase” refers to an enzyme that catalyzes the polymerization of nucleotides. “DNA polymerases” catalyze the polymerization of deoxyribonucleotides, and may include DNA-dependent DNA polymerases. Known DNA polymerases include, for example, Pyrococcus furiosus (Pfu) DNA polymerase, E. coli DNA polymerase I, T7 DNA polymerase and Thermus aquaticus (Taq) DNA polymerase, among others.

DNA polymerases also may include RNA-dependent DNA polymerases, which are called reverse-transcriptases and are associated with retroviruses. Reverse transcriptases may include the reverse transcriptase of murine leukemia viruses and engineered variants thereof utilized commercially in biotechnology fields.

“RNA polymerases” or “RNAPs” catalyze the polymerization of ribonucleotides and include DNA-dependent-RNA-polymerases. Known examples of DNA-dependent-RNA polymerases include, for example, RNA polymerases of bacteriophages (e.g. T3 RNA polymerase, T7 RNA polymerase, SP6 RNA polymerase, Syn5 RNA polymerase), and E. coli RNA polymerase, among others.

RNA polymerases may include RNA-dependent-RNA-polymerases. All RNA viruses that do not have a DNA stage of replication (e.g., as do the retroviruses) encode an RNA-dependent RNA polymerase.

The polymerase activity of DNA polymerases and RNA polymerases can be determined by means well known in the art. For example, polymerase activity may be determined via rate of incorporation of a labeled nucleotide (e.g, labeled dATP, dCTP, dGTP, or dTTP; or labeled ATP, CTP, GTP, or UTP). Labels may include radiolabels, fluorescent labels, and the like.

Stabilization and Preservation of In Vitro Transcription Reactions Through Lyophilization

The disclosed subject matter relates to compositions, systems, kits, and methods that typically comprise and/or utilize a lyophilized composition comprising components for performing in vitro transcription. The disclosed lyophilized compositions comprise components for performing transcription in vitro and typically are prepared by lyophilizing an aqueous composition comprising: (i) the components for performing transcription in vitro; and (ii) a non-reducing polysaccharide at a concentration of at least about 40 mM and/or a sugar alcohol at a concentration of at least about 40 mM.

Suitable non-reducing polysaccharides for preparing the disclosed lyophilized compositions may include, but are not limited to, non-reducing disaccharides. In some embodiments, suitable non-reducing polysaccharides may include, but are not limited to, sucrose, trehalose, maltoriose, raffinose, or a mixture thereof.

The non-reducing polysaccharide typically is present in the aqueous composition utilized to prepare the lyophilized composition at a suitable concentration (e.g., a suitable concentration for stabilizing and/or preserving an in vitro RNA transcription reaction). In some embodiments, the non-reducing polysaccharide is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

Suitable sugar alcohols for preparing the disclosed lyophilized compositions may include, but are not limited to, 6-carbon sugar alcohols, 5-carbon sugar alcohols, or a mixture thereof. In some embodiments, suitable sugar alcohols may include, but are not limited to, mannitol (e.g., D-mannitol), sorbitol, xylitol, or a mixture thereof.

The sugar alcohol typically is present in the aqueous composition utilized to prepare the lyophilized composition at a suitable concentration (e.g., a suitable concentration for stabilizing and/or preserving an in vitro RNA transcription reaction). In some embodiments, the sugar alcohol is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

In some embodiments, the lyophilized compositions are prepared from aqueous compositions comprising a relatively low concentration of glycerol. In some embodiments, the lyophilized compositions are prepared from aqueous compositions comprising no more than 5% (v/v) glycerol, preferably no more than 4%, 3%, 2%, or 1% (v/v) glycerol.

The disclosed lyophilized compositions are prepare from aqueous compositions comprising one or more components for performing transcription in vitro. Components for performing transcription in vitro may include, but are not limited to: (i) RNA polymerases; (ii) DNA transcription templates; (iii) nucleotide triphosphates (e.g. ATP, GTP, CTP, UTP or mixtures thereof); (iv) buffering agents (e.g. Tris); (v) salts (e.g., NaCl); (vi) metal cations (e.g., divalent metal cations such as Mg++), (vii) reducing agents (e.g., dithiothreitol (DTT)); (viii) polyamines (e.g., spermidine); (ix) RNase inhibitors; (x) inorganic phosphatases; (xi) albumin (e.g. bovine serum albumin (BSA); and/or (xii) purified transcription factors (e.g., see U.S. Provisional Application No. 62/758,242, filed on Nov. 8, 2018, the content of which is included as an Appendix and is incorporated herein by reference in its entirety).

The lyophilized compositions disclosed herein may include an RNA polymerase. In some embodiments, suitable RNA polymerases include DNA-dependent RNA polymerases, such as, but not limited to, E. coli RNA polymerase, and bacteriophage RNA polymerases such as T7 RNA polymerase, T3 RNA polymerase, and SP6 RNA polymerase.

The disclosed lyophilized compositions may be rehydrated to prepare rehydrated compositions. Preferably, when water is added to the lyophilized compositions to prepare a rehydrated compositions, RNA transcription occurs in the rehydrated composition (e.g., where the lyophilized composition comprises all of the necessary components for performing RNA transcription).

The lyophilized compositions may include a DNA transcription templated that encodes an aptamer such as a fluorescence-activating RNA. The lyophilized compositions further may include a dye that fluoresces in the presence of the aptamer. As such, the lyophilized compositions may be rehydrated to prepare a rehydrated composition and to synthesis an encoded fluorescence-activing RNA in the rehydrated composition which causes the dye to fluoresce, signaling that RNA transcription has occurred in the rehydrated composition.

The disclosed lyophilized compositions may be utilized to detect an analyte or target molecule in an aqueous sample. In some embodiments, the methods comprise: (i) adding the aqueous sample to the lyophilized composition of any of the foregoing claims to prepare a rehydrated composition, wherein RNA transcription occurs in the rehydrated composition if the analyte is present in the aqueous sample; and (ii) detecting RNA transcription in the rehydrated composition. In some embodiments, the rehydrated composition comprises a dye that fluoresces in the presence of an aptamer and RNA transcription synthesizes the aptamer. As such, fluorescence may be detecting in the rehydrated composition, signaling that RNA transcription has occurred.

Also disclosed are methods for preparing the disclosed lyophilized compositions. The preparation methods typical comprise lyophilizing an aqueous composition comprising: (i) components for performing transcription in vitro; and (ii) a non-reducing polysaccharide at a concentration of at least about 40 mM and/or a sugar alcohol at a concentration of at least about 40 mM.

Suitable non-reducing polysaccharides for the preparation methods may include, but are not limited to, sucrose, trehalose, maltoriose, raffinose, or a mixture thereof. In the disclosed preparation methods, preferably the non-reducing polysaccharide is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

Suitable sugar alcohols for the disclosed preparation methods may include, but are not limited to mannitol (e.g., D-mannitol), sorbitol, xylitol, or a mixture thereof. In the disclosaed preparation methods, preferably the sugar alcohol is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

In the disclosed preparation methods, preferably the aqueous composition utilized to prepare the lyophilized composition comprises a relative low amount of glycerol. Preferably, the aqueous composition comprises no more than 5% (v/v) glycerol, preferably no more than 4%, 3%, 2%, or 1% (v/v) glycerol.

In the disclosed preparation methods, preferably the aqueous composition utilized to prepare the lyophilized composition comprises a relative low amount of organic solvent, such as dimethyl sulfoxide (DMSO). Preferably, the aqueous composition comprises no more than 5% (v/v) DMSO (no more than 2 mM DMSO), preferably no more than 4%, 3%, 2%, or 1% (v/v) DMSO (no more than 1.6 mM, 1.2 mM, 0.8 mM 0.6 mM, 0.4 mM, or 0.2 mM DMSO).

In the disclosed preparation methods, the aqueous composition utilized to prepare the lyophilized composition may be present in a tube and lyophilized in the tube in order to prepare a tube comprising the lyophilized composition. The preparation methods may include purging the tube of atmospheric gas (e.g., using an inert gas such as argon), optionally placing the purged tube into a light-protective package, and optionally vacuum sealing the package. Light-protective packages are known in the art and may protect the contents of packaging from exposure to light such as ultraviolet (UV) light. The package comprising the tube may be stored in a relative cool area (e.g., <20° C.) away from sunlight.

Detection of Analytes and Target Molecules Using Regulated In Vitro Transcription of Lyophilized, Rehydrated Reaction Mixtures

The disclosed compositions, systems, kits, and methods may relate to detection of analytes and target molecules using regulated in vitro transcription of lyophilized, rehydrated reaction mixtures. The disclosed compositions, systems, kits, and methods may include and may utilize components as described herein.

The disclosed compositions, systems, kits, and methods may be utilized to detect an analyte or a target molecule in a sample. In some embodiments, the disclosed compositions, systems, kits, and methods comprise or utilize one or more components selected from: (a) an RNA polymerase; (b) an allosteric transcription factor (aTF), wherein the analyte or target molecule is a ligand to which the aTF binds; (c) an engineered transcription template; or a combination thereof. The transcription template typically comprises a promoter sequence for the RNA polymerase and an operator sequence for the aTF. The promoter sequence and operator sequence are operably linked to a sequence encoding an RNA, wherein the aTF modulates transcription of the encoded RNA when the aTF binds the analyte or target molecule as a ligand. The RNA that is transcribed from the transcription template typically binds to a reporter molecule, and the RNA binding to the reporter molecule results in a detectable signal being generated, thereby indicating that the analyte or target molecule is present.

In some embodiments of the disclosed compositions, systems, or kits, the transcribed RNA binds to the reporter molecule which RNA binding generates a detectable signal. Suitable reporter molecules may include fluorescence-activated dyes (e.g., dyes activated by an RNA aptamer as described) or fluorescently labeled double-stranded nucleotide molecules (e.g., fluorescently double-stranded DNA molecules as described herein).

In other embodiments of the disclosed compositions, systems, or kits, the compositions, systems, or kits further comprise a second engineered transcription template, in which the second engineered transcription template comprises a promoter sequence for the RNA polymerase operably linked to a sequence encoding a second RNA. In these embodiments, the second RNA binds to the reporter molecule which second RNA binding generates a detectable signal (e.g., where the reporter molecule is fluorescence-activated dye as described or a fluorescently labeled double-stranded nucleotide molecule as described herein)., and the RNA transcribed from the first engineered transcription template, namely the first RNA, interacts with the second RNA and interferes with the detectable signal generated by the second RNA binding to the reporter molecule (e.g., as a kleptamer).

Suitable RNA polymerases for inclusion or use in the disclosed compositions, systems, kits, and methods may include, but are not limited to, RNA polymerases derived from bacteriophages. Suitable RNA polymerases may include but are not limited to T7 RNA polymerase, T3 RNA polymerase, SP6 RNA polymerase, and Syn5 RNA polymerase. Suitable RNA polymerases may include engineered RNA polymerases as contemplated herein.

In the disclosed compositions, systems, kits, and methods, the allosteric transcription factor (aTF) modulates transcription from the engineered transcription template. In some embodiments, the aTF modulates transcription from the engineered transcription template when the aTF binds the operator sequence. In some embodiments, the aTF represses transcription from the engineered transcription template when the aTF binds the operator sequence. In other embodiments, the aTF activates, derepresses, and/or augments transcription from the engineered transcription template when the aTF binds the operator sequence.

In the disclosed compositions, systems, kits, and methods, the allosteric transcription factor (aTF) binds the analyte or target molecule as a ligand. In some embodiments, in the absence of the analyte or target molecule as a ligand the aTF binds to the operator sequence, and/or in the presence of the analyte or target molecule as a ligand the aTF does not bind to the operator sequence or binds to the operator sequence at a lower affinity than in the absence of the analyte or target molecule as a ligand. In other embodiments, in the presence of the analyte or target molecule as a ligand the aTF binds to the operator sequence, and/or in the absence of the analyte or target molecule as a ligand the aTF does not bind to the operator sequence or binds to the operator sequence at a lower affinity than in the presence of the analyte or target molecule as a ligand.

Allosteric transcription factors (aTFs) are known in the art. Suitable aTFs for the disclosed compositions, systems, kits, and methods may include, but are not limited to prokaryotic aTFs. Suitable aTFs may include but are not limited to TetR, MphR, QacR, OtrR, CtcS, SAR2349, MobR, and SmtB. The TetR family of aTFs include TetR, MphR, and QacR. The MarR family of aTFs include OtrR, CtcS, SAR2349, and MobR. Suitable ATF may also include the ArsR/SmtB family of ATFs.

Suitable aTFs may include engineered aTFs. For example an engineered aTF is a non-naturally occurring aTF having an amino acid sequence which has been engineered to include one or more of an insertion, a deletion, or a substitution relative to the amino acid sequence of a naturally occurring or wild-type aTF.

In some embodiments of the disclosed compositions, systems, kits, and methods, the analyte or target molecule that is a ligand for the aTF is a member of the tetracycline-family of antibiotics. Suitable analytes/target molecules as ligands for the aTF may include, but are not limited to tetracycline, anhydrotetracyline, oxytetracycline, chlortetracycline, and doxycycline.

In some embodiments of the disclosed systems and methods, the target molecule that is the ligand for the aTF is a member of the macrolide-family of antibiotics. Suitable target molecules/ligands for the aTF may include, but are not limited to erythromycin, azithromycin, and clarithromycin.

In some embodiments of the disclosed compositions, systems, kits, and methods, the analyte or target molecule that is a ligand for the aTF is a quaternary amine or salts thereof. Suitable quaternary amines may include but are not limited to alkyldimethylbenzylammonium salts.

In some embodiments of the disclosed compositions, systems, kits, and methods, the analyte that is a ligand for the aTF is a metal or a cation thereof. Suitable metals or cations thereof may include but are not limited to heavy metals and cations thereof. Suitable metals or cations thereof may include but are not limited to Zn, Pb, Cu, Cd, Ni, As, Mn (or Zn²⁺, Pb²⁺, Cu⁺, Cu²⁺, Cd²⁺, Ni²⁺, As³⁺, As⁵⁺, and Mn²⁺).

In some embodiments of the disclosed compositions, systems, kits, and methods, the analyte that is a ligand for the aTF is selected from salicylate, 3-hydroxy benzoic acid, narigenin, uric acid.

In the disclosed compositions, systems, kits, and methods, the RNA that is transcribed from the engineered transcription template typically binds to a reporter molecule, and the RNA binding to the reporter molecule results in a detectable signal being generated. Suitable transcribed RNAs for the disclosed compositions, systems, kits, and methods may include but are not limited to fluorescence-activating aptamers. Suitable transcribed RNAs may include, but are not limited to, Malachite Green aptamer, Mango aptamer, and the Spinach/Broccoli family of aptamers. Suitable transcribed RNAs may include, but are not limited to a three-way junction dimeric Broccoli (3WJdB) aptamer.

In some embodiments of the disclosed compositions, systems, kits, and methods, the compositions, systems, kits, and methods include or utilize (d) a dye, wherein the transcribed RNA is an aptamer that binds and activates the fluorescence of the dye (e.g., by forming a fluorescent complex) to generate the detectable signal. Suitable dyes that are activated by the transcribed aptamer may include but are not limited to 4-hydroxybenzlidene imidazolinone (HBI)-derivative dye, such as (5Z)-5-[(3,5-Difluoro-4-hydroxyphenyl)methylene]-3,5-dihydro-2,3-dimethyl-4H-Imidazol-4-one, (Z)-4-(3,5-Difluoro-4-hydroxybenzylidene)-1,2-dimethyl-1H-imidazol-5(4H)-one (DFHBI); (5Z)-5-[(3,5-Difluoro-4-hydroxyphenyl)methylene]-3,5-dihydro-2-methyl-3-(2,2,2-trifluoroethyl)-4H-imidazol-4-one (DFHBI-1T); 3,5-difluoro-4-hydroxybenzylidene imidazolinone-2-oxime (DFHO), thiazole orange dyes (e.g., TO1-Biotin), and Malachite Green.

In the disclosed compositions, systems, kits, and methods, the RNA that is transcribed from the engineered transcription template typically binds to a reporter molecule, and the RNA binding to the reporter molecule results in a detectable signal being generated. In some embodiments of the disclosed compositions, systems, kits, and methods, the reporter molecule is a fluorescently labeled double-stranded nucleic acid (e.g., which functions as an output gate) comprising a fluorophore and a quencher that quenches the fluorophore in the fluorescently labeled double-stranded nucleic acid. In these embodiments, the RNA that is transcribed from the engineered transcription template displaces one of the strands of the fluorescently labeled double-stranded nucleic acid which results in dequenching of the fluorophore to generate the detectable signal.

Suitable reporter molecules may include but are not limited to fluorescently labeled double-stranded DNA molecules (e.g., which function as an output gate) comprising a top strand having a fluorophore conjugated at its 3′-end and a bottom strand having a quencher conjugated at its 5′ end that quenches the fluorophore in the fluorescently labeled double-stranded DNA molecule. In these embodiments, the RNA that is transcribed from the engineered transcription template comprises a sequence that is complementary to the full length of the top strand and the transcribed RNA displaces the bottom strand which results in dequenching of the fluorophore to generate the detectable signal. Typically these reporter molecules are configured such that, the top strand is longer than the bottom strand (e.g., by about 1, 2, 3, 4, 5, 6, 7, 8, 9, 10, 11, 12, 13, 14, 15 nucleotides or more). In this configuration, displacement of the bottom strand by the transcribed RNA is thermodynamically favored because the transcribed RNA comprises a sequence that is complementary to the full length of the top strand, which permits additional base-pairing between the transcribed RNA and the top strand that is not presented between the top strand and the bottom strand. Optionally, the disclosed systems and methods further may comprise a non-labeled double-stranded DNA molecule (e.g., which functions as a threshold gate) comprising a top strand that comprises a nucleotide sequence that is identical to the nucleotide sequence of the top strand of the labeled double-stranded DNA molecule. Typically, the top strand of the non-labeled double-stranded DNA molecule is longer than the bottom strand of the non-labeled double-stranded DNA molecule (e.g., by about 1, 2, 3, 4, 5, 6, 7, 8, 9, 10, 11, 12, 13, 14, 15 nucleotides or more). Optionally, the bottom strand of the non-labeled double-stranded DNA molecule is shorter in length than the length of the bottom strand of the fluorescently labeled double-stranded DNA molecule (e.g., by about 1, 2, 3, 4, 5, 6, 7, 8, 9, 10, 11, 12, 13, 14, 15 nucleotides or more), such that displacement of the bottom strand of the non-labeled double-stranded DNA molecule is favored thermodynamically versus displacement of the bottom strand of the fluorescently labeled double-stranded DNA molecule.

In some embodiments of the disclosed compositions, systems, kits, and methods, multiple aTFs and/or multiple engineered transcription templates may be included and/or utilized. For example, multiple aTFs and/or multiple engineered transcription templates may be included and/or utilized in order to create logic gates.

The disclosed compositions, systems, kits, and methods may be utilized to detect one or more analytes or target molecules (i.e., multiple analytes or target molecules) in a sample. In some embodiments, the disclosed compositions, systems, kits, and methods may include or utilize: (a) one or more RNA polymerases; and (b) two or more aTFs; and/or (c) two or more engineered transcription templates. In some embodiments, the compositions, systems, kits, and methods may include or utilize: (a) one or more RNA polymerases; (b)(i) a first allosteric transcription factor (aTF), wherein one or more of the analytes or target molecules is a ligand to which the first aTF binds; (b)(ii) a second allosteric transcription factor (aTF), wherein one or more of the analytes or target molecules is a ligand to which the second aTF binds; (c)(i) a first engineered transcription template, the first engineered transcription template comprising a promoter sequence for the RNA polymerase and an operator sequence for first aTF operably linked to a sequence encoding a first RNA, wherein the first aTF modulates transcription of the encoded first RNA when the first aTF binds the analyte or target molecule as a ligand; and (c)(ii) a second engineered transcription template, the second engineered transcription template comprising a promoter sequence for the RNA polymerase and an operator sequence for the second aTF operably linked to a sequence encoding a second RNA, wherein the second aTF modulates transcription of the encoded second RNA when the second aTF binds the analyte or target molecule a ligand. In these embodiments, the first transcribed RNA, the second transcribed RNA, and a reporter molecule form a complex that generates a detectable signal. In some embodiment, the first transcribed RNA and the second transcribed RNA interact, for example, to form at least a partially double stranded RNA complex (e.g., an aptamer generated from split parts) which binds to the reporter molecule, where binding of the RNA complex to the reporter molecule generates a detectable signal. In some embodiments, the first transcribed RNA and the second transcribed RNA interact to form a fluorescence-activating aptamer, which may include but is not limited to a Split-Broccoli aptamer. The fluorescence-activating aptamer formed from the first transcribed RNA and the second transcribed RNA may bind and activate the fluorescence of a dye (e.g., by forming a fluorescent complex) to generate the detectable signal. In some embodiments, the first transcribed RNA and the second transcribed RNA interact to inhibit the formation of a fluorescence-activating aptamer, which may include but is not limited to a 3WJdB aptamer. In some embodiments, the first transcribed RNA interacts with the aTF and inhibits its ability to bind to its operator, thus increasing the production of the second RNA which may include but is not limited to a fluorescence-activating aptamer. Suitable dyes that are activated by the transcribed aptamer may include but are not limited to 4-hydroxybenzlidene imidazolinone (HBI)-derivative dye, such as (5Z)-5-[(3,5-Difluoro-4-hydroxyphenyl)methylene]-3,5-dihydro-2,3-dimethyl-4H-Imidazol-4-one, (Z)-4-(3,5-Difluoro-4-hydroxybenzylidene)-1,2-dimethyl-1H-imidazol-5(4H)-one (DFHBI); (5Z)-5-[(3,5-Difluoro-4-hydroxyphenyl)methylene]-3,5-dihydro-2-methyl-3-(2,2,2-trifluoroethyl)-4H-imidazol-4-one (DFHBI-1T); 3,5-difluoro-4-hydroxybenzylidene imidazolinone-2-oxime (DFHO), thiazole orange dyes (e.g., TO1-Biotin), and Malachite Green.

The compositions, systems, kits, and methods disclosed herein further may include or utilize additional components, such as additional components for performing RNA transcription. Additional components may include but are not limited to one or more of ribonucleoside triphosphates, an aqueous butter system that includes a reducing agent such dithiothreitol (DTT), divalent cations such as Mg⁺⁺, spermidine, an inorganic pyrophosphatase, an RNase inhibitor, crowding agents, and monovalent salts (e.g., NaCl and K-glutamate).

The components of the disclosed compositions, systems, kits, and methods may be mixed. For example, the components of the disclosed compositions, systems, kits, and methods may be mixed as an aqueous solution and/or may be dried or lyophilized to prepare a dried mixture which may be rehydrated and/or reconstituted (e.g., to perform the methods disclosed herein).

The disclosed compositions, systems, and kits, and the components thereof may be utilized in methods for detecting an analyte or target molecule in a sample (e.g., by performing an RNA transcription reaction). The methods may include contacting one or more components of the disclosed compositions, systems, and kits with the sample and detecting a detectable signal, thereby detecting the analyte or target molecule in the sample.

### Illustrative Embodiments

The following embodiments are illustrative and should not be interpreted to limit the scope of the claimed subject matter.

Embodiment 1. A lyophilized composition comprising components for performing transcription in vitro prepared by lyophilizing an aqueous composition comprising: (i) the components for performing transcription in vitro; and (ii) a non-reducing polysaccharide at a concentration of at least about 40 mM and/or a sugar alcohol at a concentration of at least about 40 mM.

Embodiment 2. The lyophilized composition of embodiment 1, wherein the non-reducing polysaccharide is a non-reducing disaccharide.

Embodiment 3. The lyophilized composition of embodiment 1 or 2, wherein the non-reducing polysaccharide is selected from sucrose, trehalose, maltoriose, raffinose, or a mixture thereof.

Embodiment 4. The lyophilized composition of any of the foregoing embodiments, wherein the non-reducing polysaccharide is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

Embodiment 5. The lyophilized composition of any of the foregoing embodiments, wherein the sugar alcohol is a 6-carbon sugar alcohol, a 5-carbon sugar alcohol, or a mixture thereof.

Embodiment 6. The lyophilized composition of any of the foregoing embodiments, wherein the sugar alcohol is selected from mannitol (e.g., D-mannitol), sorbitol, xylitol, or a mixture thereof.

Embodiment 7. The lyophilized composition of any of the foregoing embodiments, wherein the sugar alcohol is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

Embodiment 8. The lyophilized composition of any of the foregoing embodiments, wherein the aqueous composition comprises no more than 5% (v/v) glycerol, preferably no more than 4%, 3%, 2%, or 1% (v/v) glycerol.

Embodiment 9. The lyophilized composition of any of the foregoing embodiments, wherein the aqueous composition comprises one or more components for performing transcription in vitro selected from (i) an RNA polymerase; (ii) a DNA transcription template; (iii) nucleotide triphosphates; and/or (iv) a buffering agent (e.g. Tris).

Embodiment 10. The lyophilized composition of embodiment 9, wherein the aqueous composition further comprises one or more components selected from a salt (e.g., NaCl), a metal ion (e.g., a divalent metal cation such as Mg++), a reducing agent (e.g., DTT), a polyamine (e.g., spermidine), an RNase inhibitor, inorganic pyrophosphatase, bovine serum albumin, and/or purified transcription factors.

Embodiment 11. The lyophilized composition of embodiment 9 or 10, wherein the RNA polymerase is selected from E. coli RNA polymerase, T7 RNA polymerase, T3 RNA polymerase, and SP6 RNA polymerase.

Embodiment 12. The lyophilized composition of embodiment 9 or 10, wherein the DNA transcription template encodes an aptamer (e.g., a fluorescence-activating RNA).

Embodiment 13. The lyophilized composition of embodiment 12, wherein the aqueous composition further comprises a dye that fluoresces in the presence of the aptamer.

Embodiment 14. The lyophilized composition of any of the foregoing embodiments, wherein after water is added to the lyophilized composition to prepare a rehydrated composition, RNA transcription occurs in the rehydrated composition.

Embodiment 15. The lyophilized composition of any of the foregoing claims, wherein the aqueous composition does not comprise more than about 5% (v/v) organic solvent such as DMSO (preferably no more than 4%, 3%, 2%, or 1% (v/v)).

Embodiment 16. The lyophilized composition of any of the foregoing claims, wherein the lyophilized composition is present in a tube, optionally a tube which has been purged of atmospheric gas (e.g., via use of an inert gas such as argon), optionally wherein the tube is present in a light-protective package (e.g., a package that protects its contents from ultraviolet (UV) light, and optionally wherein the package has been vacuum sealed.

Embodiment 17. A method for detecting an analyte in an aqueous sample, the method comprising: (i) adding the aqueous sample to the lyophilized composition of any of the foregoing embodiments to prepare a rehydrated composition, wherein RNA transcription occurs in the rehydrated composition if the analyte is present in the aqueous sample; and (ii) detecting RNA transcription in the rehydrated composition.

Embodiment 18. The method of embodiment 17, wherein the rehydrated composition comprises a dye that fluoresces in the presence of an aptamer and RNA transcription synthesizes the aptamer.

Embodiment 19. A method for preparing the lyophilized composition of any of embodiments 1-16, the method comprising lyophilizing an aqueous composition comprising: (i) components for performing transcription in vitro; and (ii) a non-reducing polysaccharide at a concentration of at least about 40 mM and/or a sugar alcohol at a concentration of at least about 40 mM.

Embodiment 20. The method of embodiment 19, wherein the non-reducing polysaccharide is selected from sucrose, trehalose, maltoriose, raffinose, or a mixture thereof, and the non-reducing polysaccharide is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

Embodiment 21. The method of embodiment 19 or 20, wherein the sugar alcohol is selected from mannitol (e.g., D-mannitol), sorbitol, xylitol, or a mixture thereof and the sugar alcohol is present in the aqueous composition at a concentration of at least about 40 mM, 60 mM, 80 mM, 100 mM, 150 mM, 200 mM, 250 mM, 300 mM, 350 mM, or 400 mM or a concentration within a range bounded by two percentage values of any of 40 mM, 60 mM, 80 mM, 100 mM, 150 mM, 200 mM, 250 mM, 300 mM, 350 mM, or 400 mM (e.g., a concentration within a range of 40-100 mM).

Embodiment 22. The method of any of embodiments 19-21, wherein the aqueous composition comprises no more than 5% (v/v) glycerol, preferably no more than 4%, 3%, 2%, or 1% (v/v) glycerol.

Embodiment 23. The method of any of embodiments 19-22, wherein the aqueous composition comprises no more than 5% (v/v) organic solvent (e.g., dimethyl sulfoxide (DMSO), preferably no more than 4%, 3%, 2%, or 1% (v/v) organic solvent.

Embodiment 24. The method of any of embodiments 19-23, wherein the aqueous composition is present in a tube and is lyophilized in the tube to prepare a tube comprising the lyophilized composition, optionally wherein the tube is purged of atmospheric gas (e.g., by applying an inert gas such as argon), and optionally wherein the tube is placed into a light-protective package (e.g., a package that protects its contents from ultraviolet (UV) light), and optionally wherein the package is vacuum sealed, and optionally wherein the package is stored at a temperature less than about 20° C. and away from light.

Embodiment 25. A lyophilized composition, system, kit, or method for detecting an analyte or a target molecule in a sample, the composition, system, kit, or method comprising and/or utilizing any of the lyophilized compositions, components, or methods of the foregoing embodiments. Optionally, the lyophilized composition, system, kit, or method comprises or utilizes one or more lyophilized components selected from: (a) an RNA polymerase; (b) an allosteric transcription factor (aTF), wherein the analyte or target molecule is a ligand to which the aTF binds; (c) an engineered transcription template; or a combination thereof. The transcription template optionally comprises a promoter sequence for the RNA polymerase and an operator sequence for the aTF. Optionally, the promoter sequence and operator sequence are operably linked to a sequence encoding an RNA, wherein the aTF modulates transcription of the encoded RNA when the aTF binds the analyte or target molecule as a ligand. Optionally, the RNA that is transcribed from the transcription template typically binds to a reporter molecule, and the RNA binding to the reporter molecule results in a detectable signal being generated, thereby indicating that the analyte or target molecule is present.

### EXAMPLES

The following examples are illustrative and should not be interpreted to limit the scope of the claimed subject matter.

### Example 1

Title—Stabilization and Preservation of In Vitro Transcription Reactions Through Lyophilization

Summary of Technology

We demonstrate that in vitro transcriptions (IVT) can be optimally stabilized through lyophilization using a combination of a non-reducing disaccharide and a sugar alcohol as lyoprotectants. These IVT reactions can be lyophilized in tubes, packaged in a light-protective, vacuum-sealed bag for long-term shelf stability, and reactivated by rehydration. We further show that regulated in vitro transcriptions can also be stabilized using this same process and formulation. (See U.S. Provisional Application No. 62/758,242, filed on Nov. 9, 2018, the content of which is included as an Appendix and is incorporated herein by reference in its entirety).

Summary of Findings

1. Freeze-drying IVT is optimal when including a nonreducing disaccharide.
a. Preferably sucrose or trehalose (disaccharides) due to availability and cost;
b. Alternatively, one could use maltotriose, raffinose, and other higher order saccharides. These are generally not preferred due to cost; and
c. Suggested concentration ranges from at least 20 mM, 30 mM, 40 mM, 50 mM, 60 mM, to up to 400 mM.

2. The combination of a nonreducing saccharide (from above) with a sugar alcohol improves the physical properties of the freeze-dried product. The subsequent freeze-dried “cake” is honeycombed, dry, and matte in appearance, and is easier to rehydrate than lyophilized reactions without a sugar alcohol.
a. Preferably mannitol (6 carbon sugar alcohol), but could also be sorbitol (another C₆) or similar.
b. Could also use xylitol (C₅) or some other sugar alcohol.
c. Suggested concentration ranges from at least 20 mM, 30 mM, 40 mM, 50 mM, 60 mM, to up to 400 mM.

3. Lyophilized IVT reactions should be prepared with minimal glycerol.
a. The proteins typically added to an IVT (RNA polymerase, RNase inhibitors, inorganic pyrophosphatase, purified transcription factors, etc.) are often prepared and stored in glycerol.
b. Dialysis to remove glycerol from reaction components helps significantly.
c. Suggested range of less than 5% (v/v) (or 6.25 g/L), preferably less than 4%, 3%, 2%, or 1% (v/v) (1.25 g/L).

4. Lyophilized IVT reactions should be prepared with minimal organic solvent, specifically dimethyl sulfoxide (DMSO).
a. The dyes typically used in IVT to activate signal from fluorescence-activating RNA aptamers (DFHBI-1T, DFHO, etc.) are often prepared and stored in organic solvents such as DMSO.
b. Suggested range of less than 5% (v/v), or 2 mM.

5. The ideal method is to assemble IVT reaction components together and then immediately transfer to a pre-chilled aluminum block to halt reaction progress. Immediately thereafter, the reaction is optionally slow-cooled to a freeze (e.g. transferring to a −80° C. freezer). The reaction is then snap-cooled in liquid nitrogen. The frozen reaction is transferred to a lyophilizer with a suitably cold condenser temperature (e.g. −85° C.) and pressure (e.g. 0.04 mBar) for >2 hours to allow primary and secondary drying of the IVT reaction.

6. The ideal method of packaging lyophilized IVT reactions is to:
a. Place the tubes containing lyophilized IVT reactions in a light-protective bag (e.g. mylar food bags). It is best to leave the caps with holes.
b. Add a dricard desiccant to the bag.
c. Purge with inert gas such as Argon or Nitrogen.
d. Immediately either vacuum seal or impulse heat-seal the bag.
e. Store it in a cool, shaded area away from direct sources of heat and sunlight.

Background

In vitro transcription (IVT) is the process by which RNA is synthesized. In its simplest form, it comprises: a DNA transcription template, an RNA polymerase, nucleotide triphosphates (NTPs), and additional cofactors that are formulated into a buffer for the reaction.

While numerous RNA polymerases can be used for IVT, including E. coli RNA polymerase, more often than not a bacteriophage polymerase is used. In practice, this typically includes the single-subunit RNA polymerase from the bacteriophages T7, T3, and SP6. Additional protein components of an IVT may include, but are not limited to, an RNase inhibitor, inorganic pyrophosphatase, bovine serum albumin, and purified transcription factors. Additional chemical components for IVT may include, but are not limited to, a buffering agent (e.g. Tris), salt (e.g. NaCl), metal ion (Mg), a reducing agent (e.g. DTT), a polyamine (e.g. spermidine).

In Vitro Transcriptional Sensors

Fluorescence-activating RNAs are genetically encoded aptamers that can bind to an otherwise non-fluorescent molecule and activate its fluorescence. These aptamers can be used as signaling outputs to report on transcriptional activity. We use a simple IVT sensor to assess the functionality of IVT reactions before and after freeze-drying. In short, this sensor comprised transcription of the 3WJdB aptamer (DOI: 10.1021/acssynbio.7b00059) in the presence of an otherwise non-fluorescent dye, DFHBI-1T (DOI: 10.1021/ja410819x). The transcribed aptamer can then bind the dye and activate its fluorescence in a concentration-dependent manner, thereby reporting on IVT activity.

Materials

All IVT reactions were performed with the following components. Typical reactions volumes were 20 μL.
1. T7 RNA polymerase (0.2 μg of a laboratory prep. Or 100-200 U of New England Biolabs #M0251).
2. Transcription buffer
   8-27 mM MgCl₂
   2 mM spermidine
   40 mM Tris-HCl pH 8
   10 mM DTT
   20 mM NaCl
3. 2.25 mM DFHBI-1T
4. Nucleotide triphosphates (NTP)
   2-10 mM of each ATP, CTP, GTP, UTP
5. 0.5-5 pmol transcription template (T7-3WJdB-T, DOI: 10.1021/acssynbio.7b00059)
   Double-stranded PCR amplification product encoding a region<100 basepairs upstream of the T7 promoter and ending with the T7 terminator was prepared and purified using a spin-column PCR purification kit.

Data

The following data compares fresh reactions (i.e. no lyophilization) to reactions that were lyophilized overnight and then rehydrated with laboratory-grade water. In summary, many reagents known to offer cryoprotection, lyoprotection, or used as excipients in freeze-dried reactions were tried. The overwhelming conclusions were that nonreducing sugars offered lyoprotection, which was further improved with the addition of a sugar alcohol.

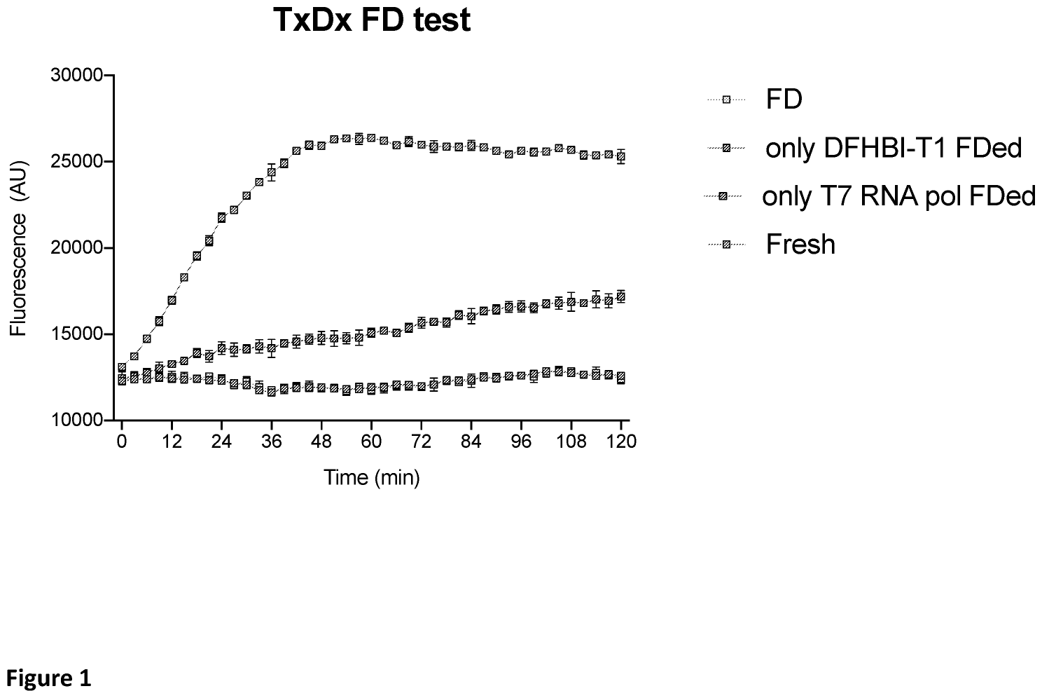

We first confirmed that activity of the IVT sensor was dependent on successful lyoprotection of the T7 RNA polymerase, rather than the dye used for the IVT sensor. In the data in FIG. 1, a fresh reaction is compared to reactions in which either the dye needed for the sensor was freeze-dried (FD), or the T7 RNA polymerase was FD. When T7 RNA polymerase was freeze-dried, it was unable to function as an IVT sensor when added to a fresh reaction. (See FIG. 1). However, the dye was able to function as an IVT sensor. (See FIG. 1).

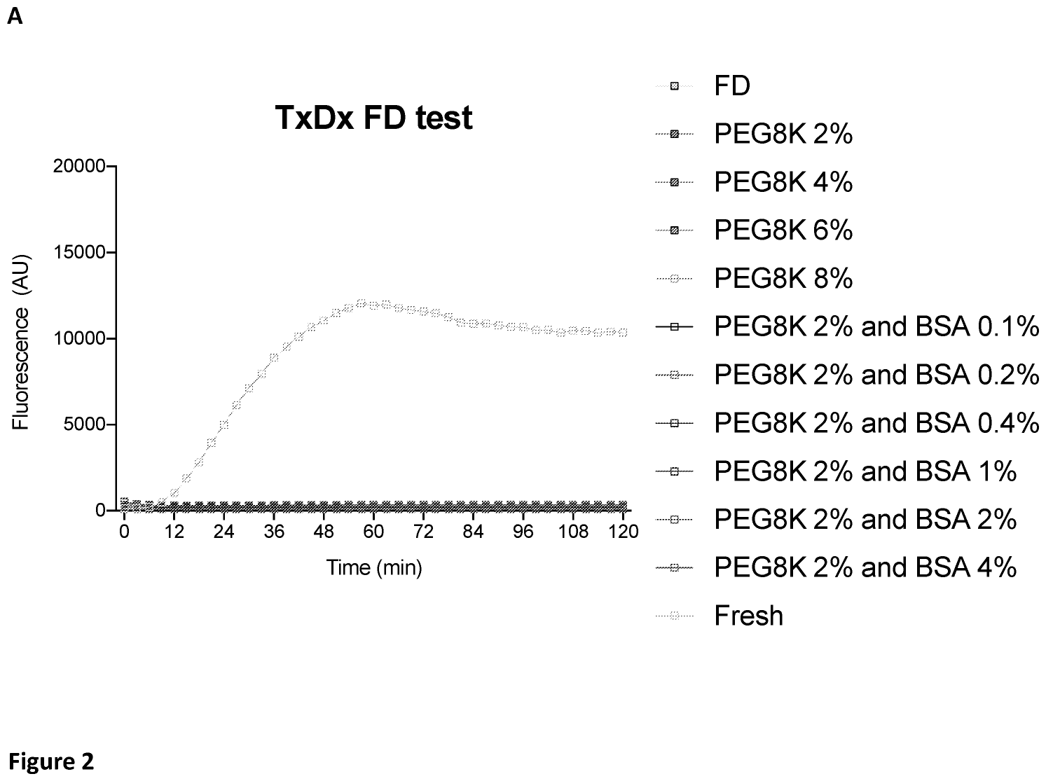

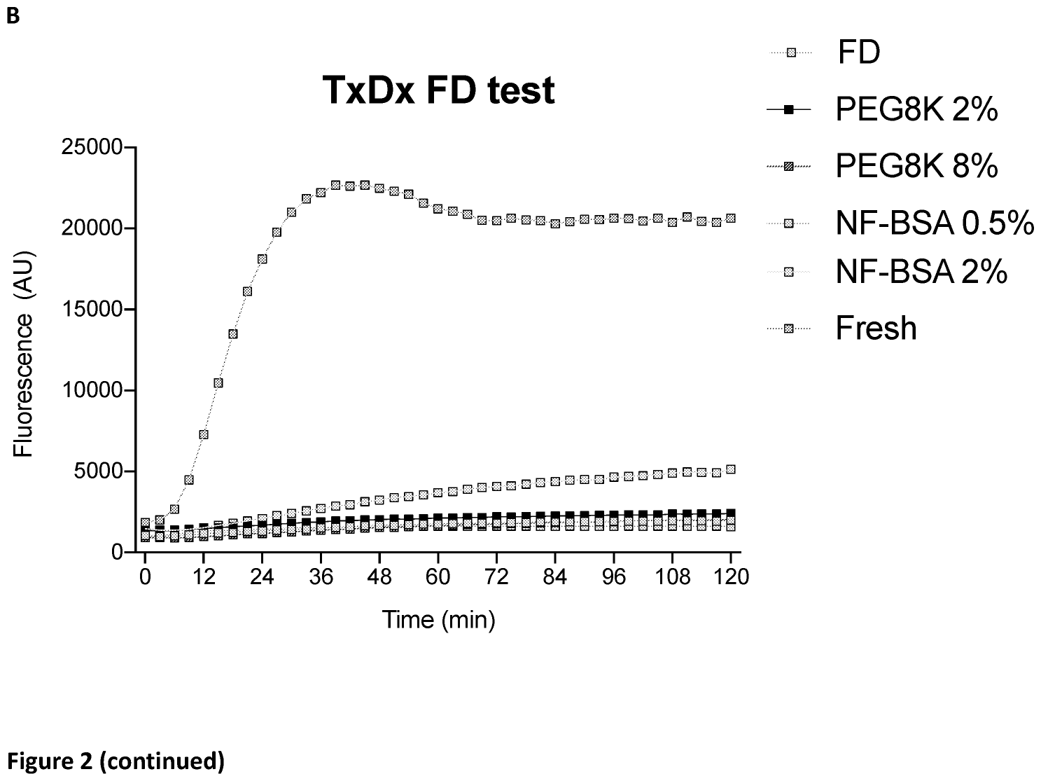

We next tested a high molecular weight compound, polyethylene glycol (8000), independently and in combination with bovine serum albumin to determine whether it could impart lyoprotection. (See FIGS. 2A and 2B). These high molecular weight molecules are often used to protect reactions from the irreversible damage caused by freezing temperatures. Neither offered suitable lyoprotection. (See FIGS. 2A and 2B).

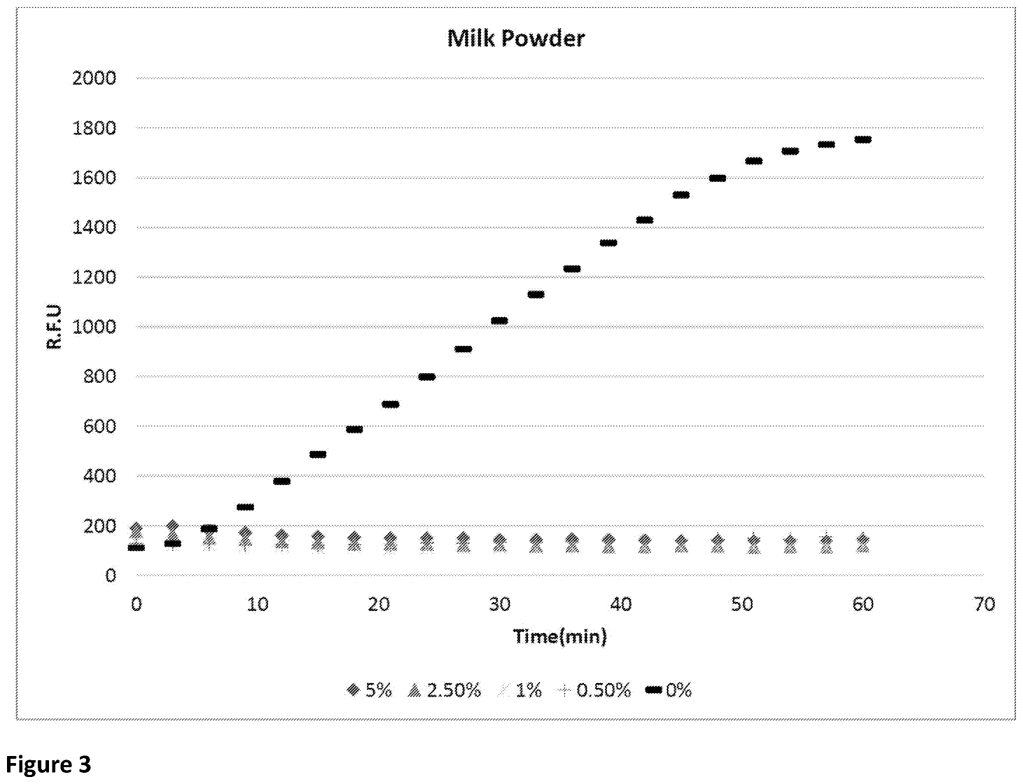

We next tested dried milk powder, which contains a rich mixture of proteins (i.e. high molecular weight compounds). (See FIG. 3). However, dried milk powder, did not offer any lyoprotection. (See FIG. 3).

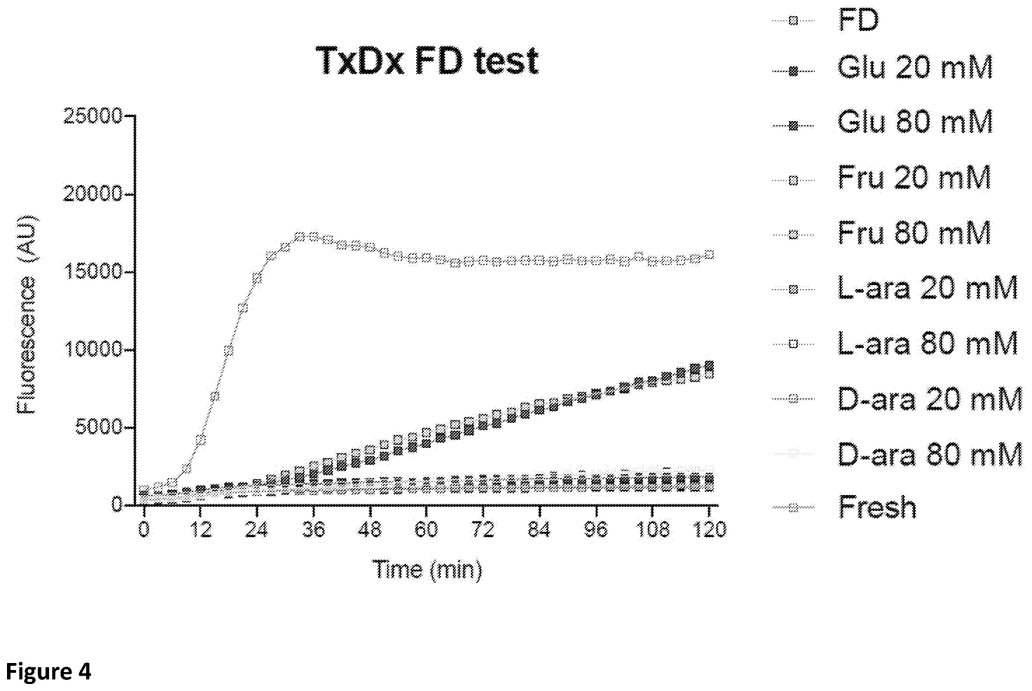

We next tested various sugars, which were added from 1M stock solutions. We found that sugars in general, and especially disaccharides, offered robust lyoprotection at >2% (20 mM), with optimal protection between 8-20% (v/v) (80-200 mM). (See FIG. 4). The following sugars were tested independently: glucose (Glu), fructose (Fru), L-arabinose (L-ara), D-arabinose (D-ara), cellobiose (Cel), lactose (Lac), maltose (Mal), sorbitol (Sor), sucrose, and trehalose. (See FIG. 4) Both fructose and glucose, simple monosaccaharide sugars with either 5 or 6 carbons, at 8% (v/v) (80 mM) offered some lyoprotection when compared to either a FD reaction without any additional components, to isomers of arabinose (L-ara, D-ara) at 2 and 8% (v/v) (or 20 mM and 80 mM, respectively), or to lowered concentrations of glucose and fructose (2% (v/v) or 20 mM). (See FIG. 4).

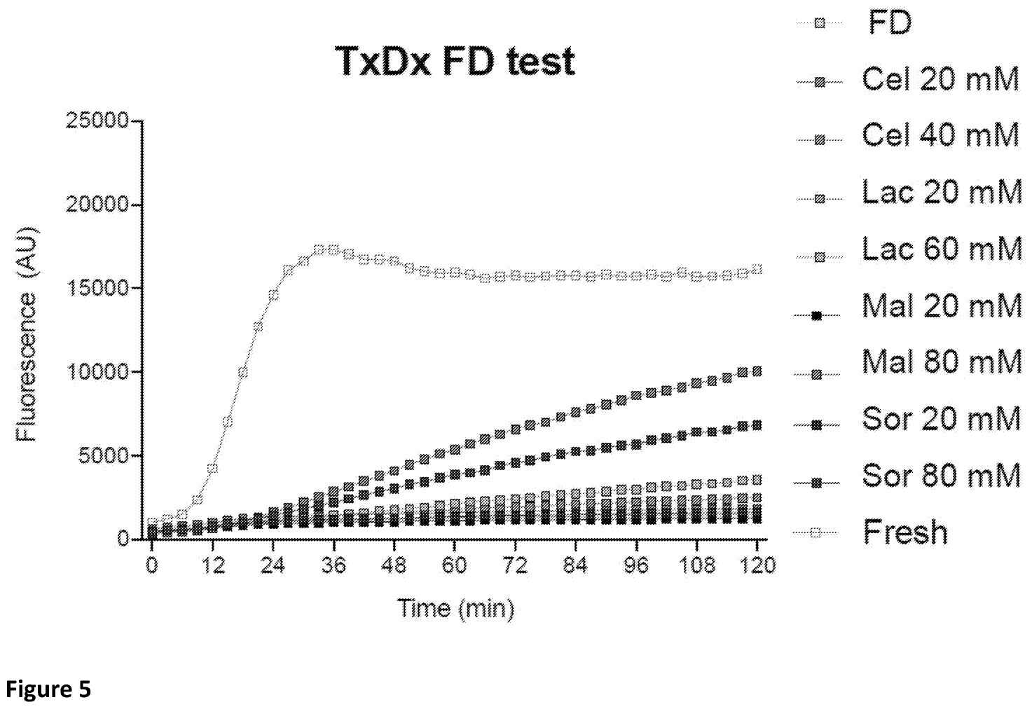

Additional sugars were tested, including cellobiose (a disaccharide comprising 2 covalently linked units of glucose), lactose (a disaccharide of glucose and galactose), maltose (another disaccharide consisting of two glucose molecules covalently linked), and sorbitol (a 6 carbon sugar alcohol). (See FIG. 5). Turanose, a reducing disaccharide, also was tested (data not shown). Only 8% (80 mM) maltose and 8% (80 mM) sorbitol offered substantial lyoprotection, and in all cases, lower percentages of the sugar resulted in reduced or no lyoprotection. (See FIG. 5).

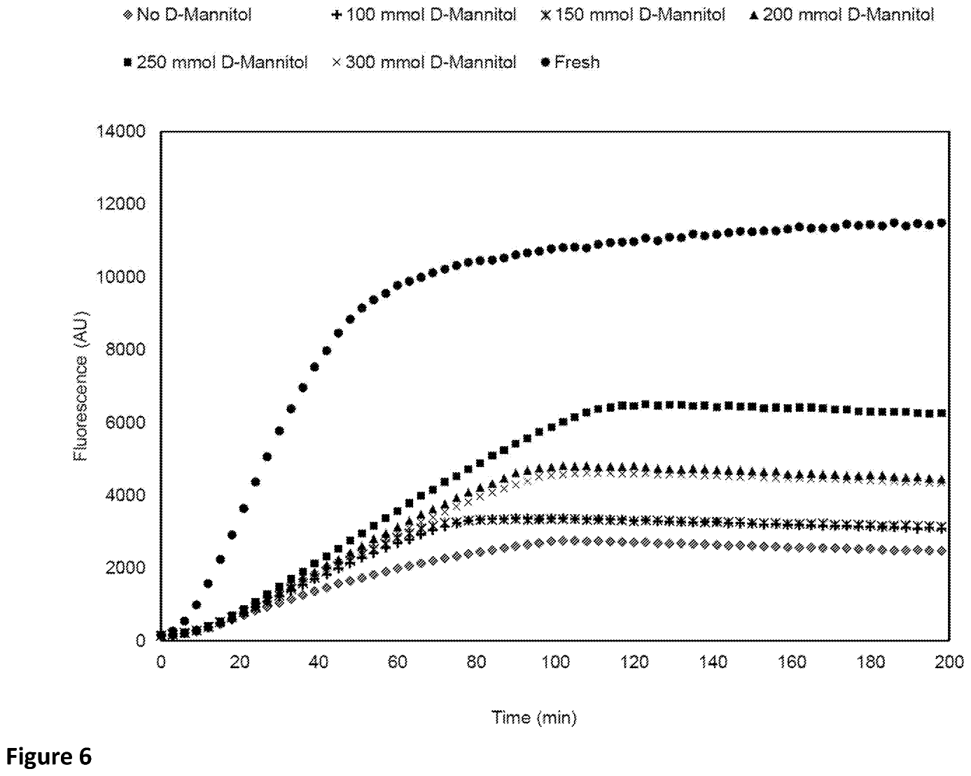

We also tested sugar alcohols. (See FIG. 6). In particular, D-mannitol as observed to exhibit lyoprotection of the IVT sensor. (See FIG. 6).

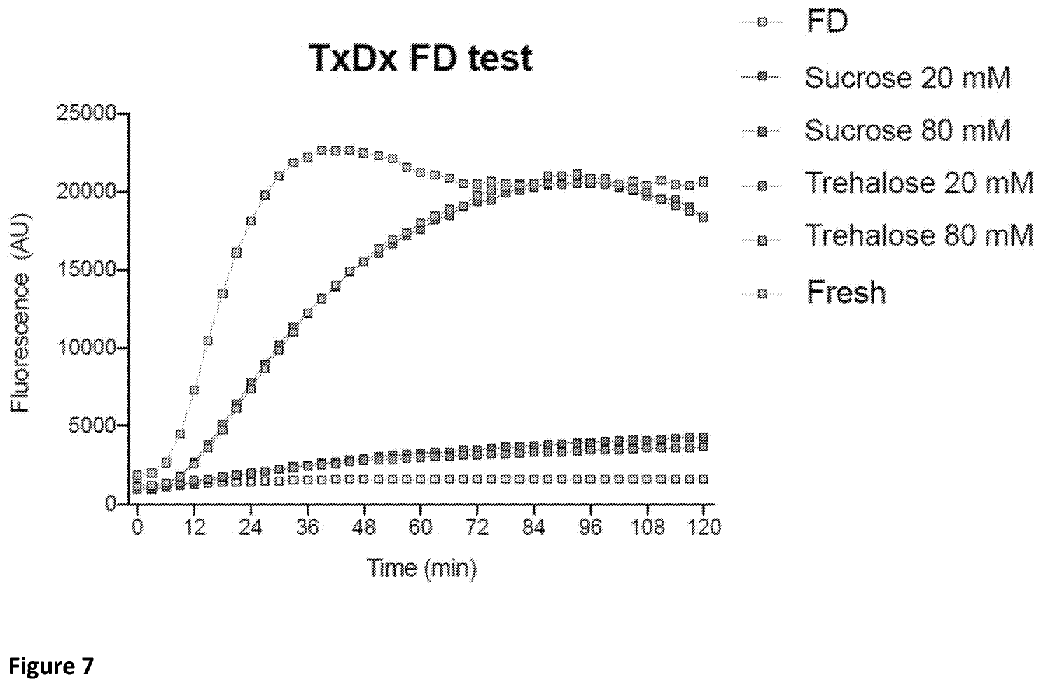

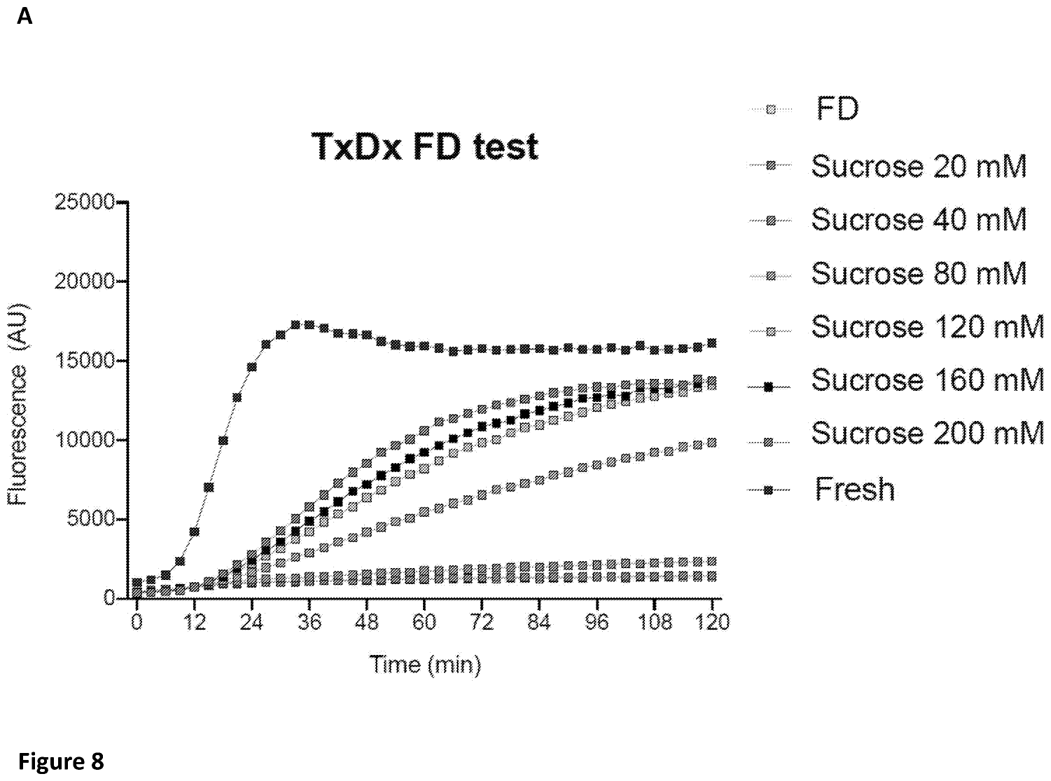

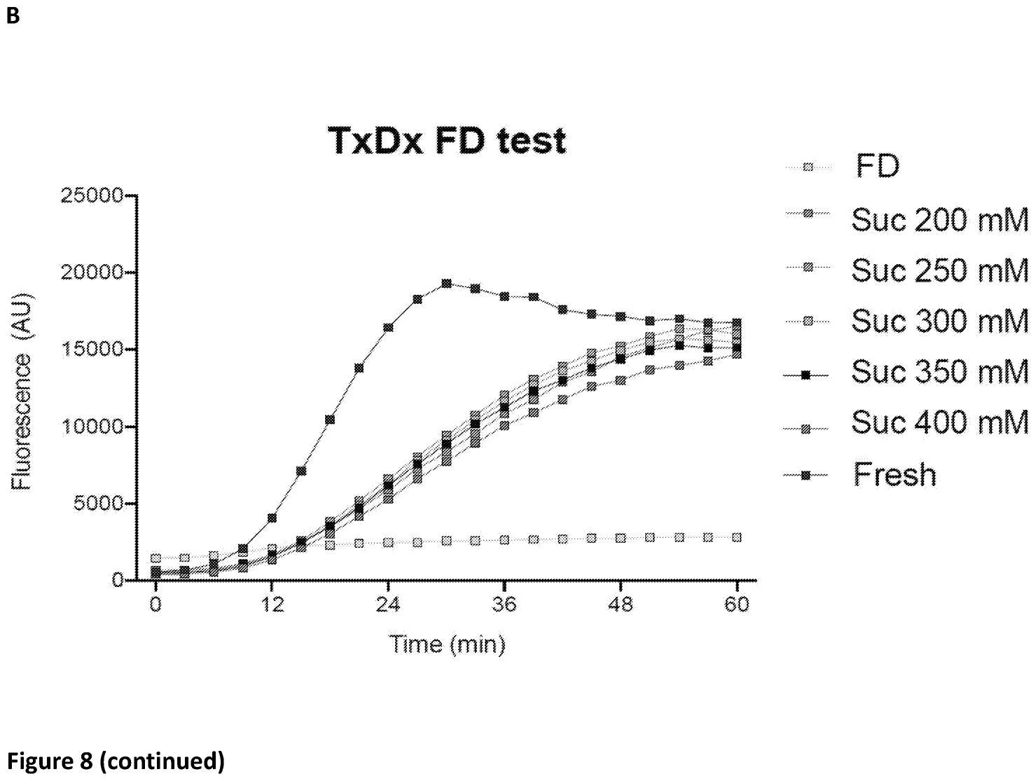

We found that both sucrose and trehalose (non-reducing disaccharides) were capable of restoring nearly all functionality (when compared to a fresh reaction) when included in the lyophilized reaction at 8% (80 mM). (See FIG. 7). The lyoprotection using sucrose or trehalose extended into high percentages (up to 40% (v/v), or 400 mM). (See FIGS. 8A and 8B).

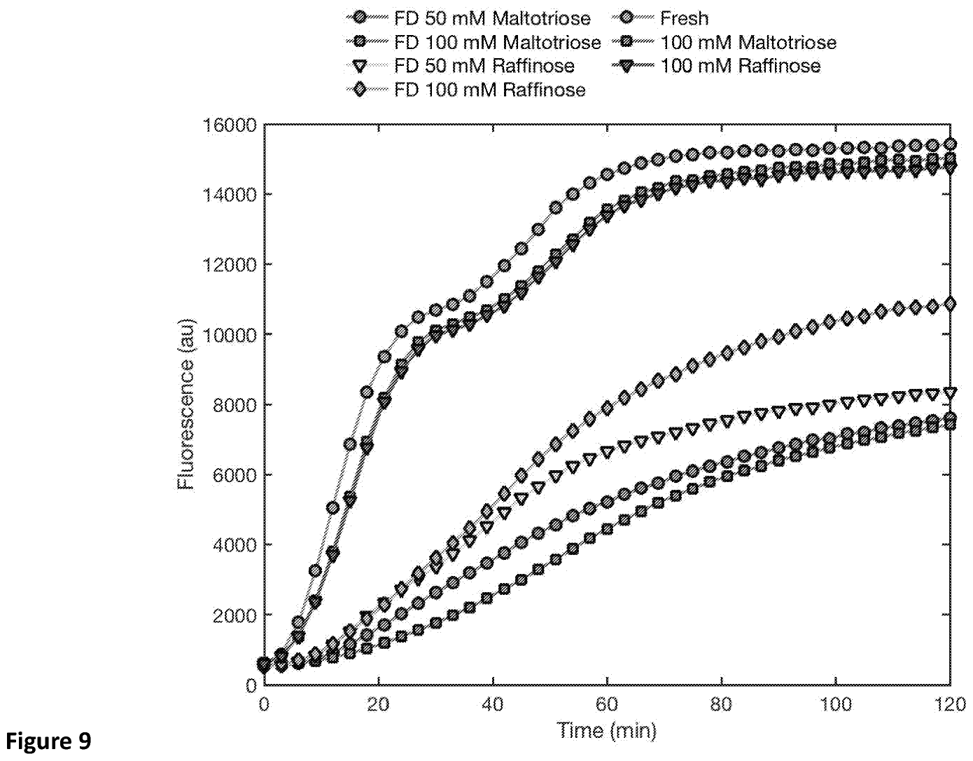

Based on the success of the non-reducing disaccharides, we tested the non-reducing trisaccharides maltotriose and raffinose. (See FIG. 9). Both trisaccharides offered lyoprotection, but were unused in further experiments due to their increased cost compared to sucrose and trehalose.

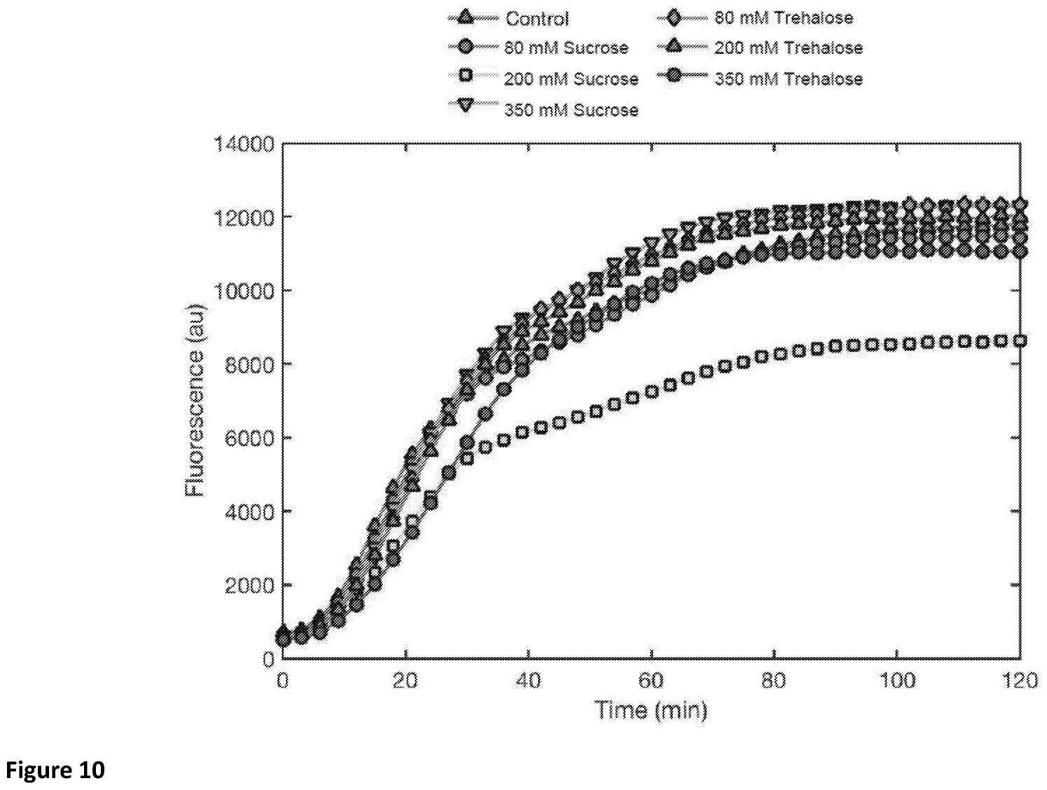

We also confirmed that the addition of sucrose or trehalose up to 35% (v/v) (350 mM) into the reaction does not have a negative impact on the functionality on the IVT sensor when tested as a fresh and not freeze-dried reaction. (See FIG. 10).

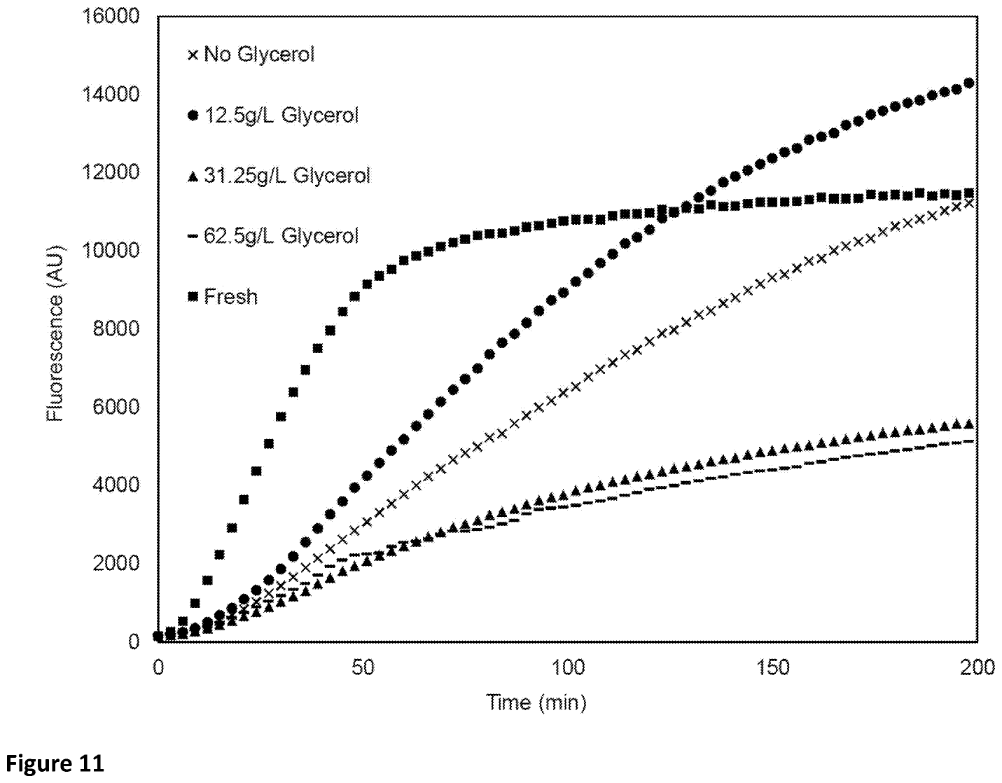

We observed that dialysis of proteins (e.g. T7 RNA polymerase) into transcription buffer without glycerol improved the reaction to levels similar to the fresh reaction. (See FIG. 11). Similarly, as the amount of glycerol was lowered, activity of the sensor increased. (See FIG. 11).

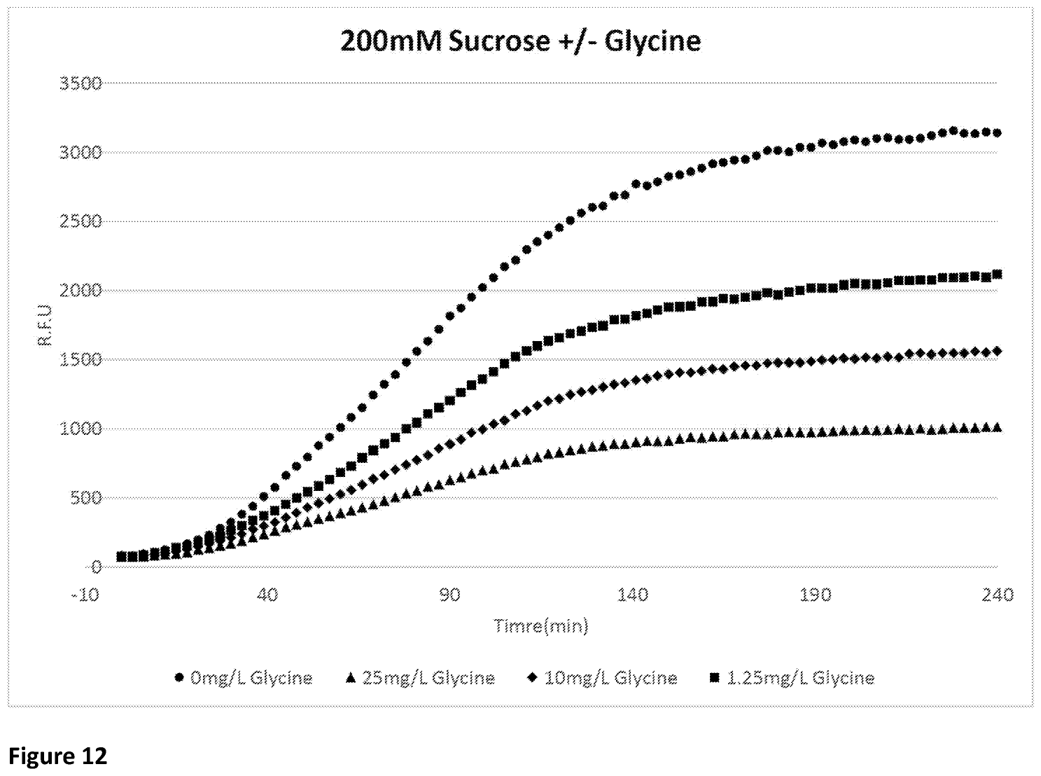

To determine if the activity of a lyophilized IVT sensor could be further improved, we next tried combinations of a nonreducing disaccharide (sucrose) at 20% (v/v) or 200 mM with additional compounds. The addition of glycine to an IVT reaction containing 20% (200 mM) sucrose did not improve lyoprotection. (See FIG. 12).

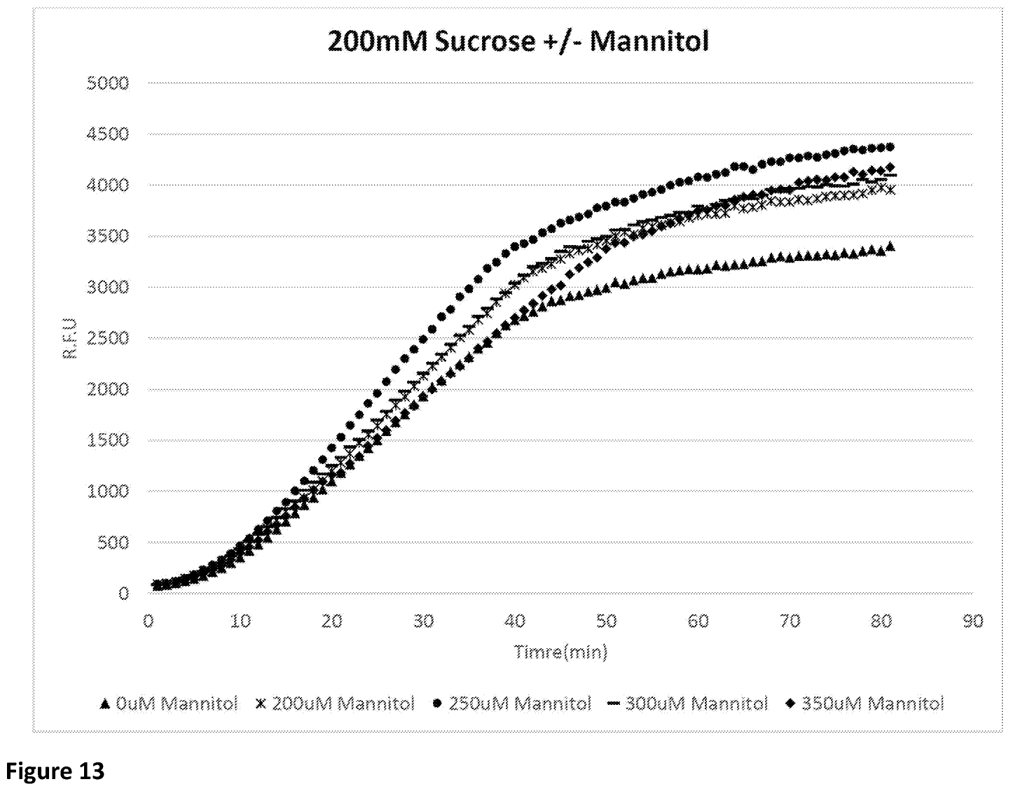

However, the addition of mannitol to an IVT reaction containing 20% (200 mM) sucrose did improve lyoprotection. (See FIG. 13).

Regulated In Vitro Transcription

We recently showed that IVT reactions can be regulated by designing a transcription template that includes a binding site for an allosteric transcription factor (aTF), and including that aTF into the reaction mixture. (See U.S. Provisional Application No. 62/758,242, filed on Nov. 9, 2018, the content of which is included as an Appendix and is incorporated herein by reference in its entirety). We demonstrated this using the fast and processive bacteriophage T7 RNA polymerase and showed that regulating the production of a fluorescence-activating RNA aptamer enables a biosensing platform in which a fluorescent output is dependent on the presence or absence of a ligand of interest. Finally, we have demonstrated that more than one DNA template can be included to serve as a RNA genetic circuitry to improve functions of a sensor (e.g. specificity, sensitivity). We provide here the data showing that this formulation can stabilize a regulated IVT after lyophilization. We provide here the data showing that this formulation can stabilize a regulated IVT.

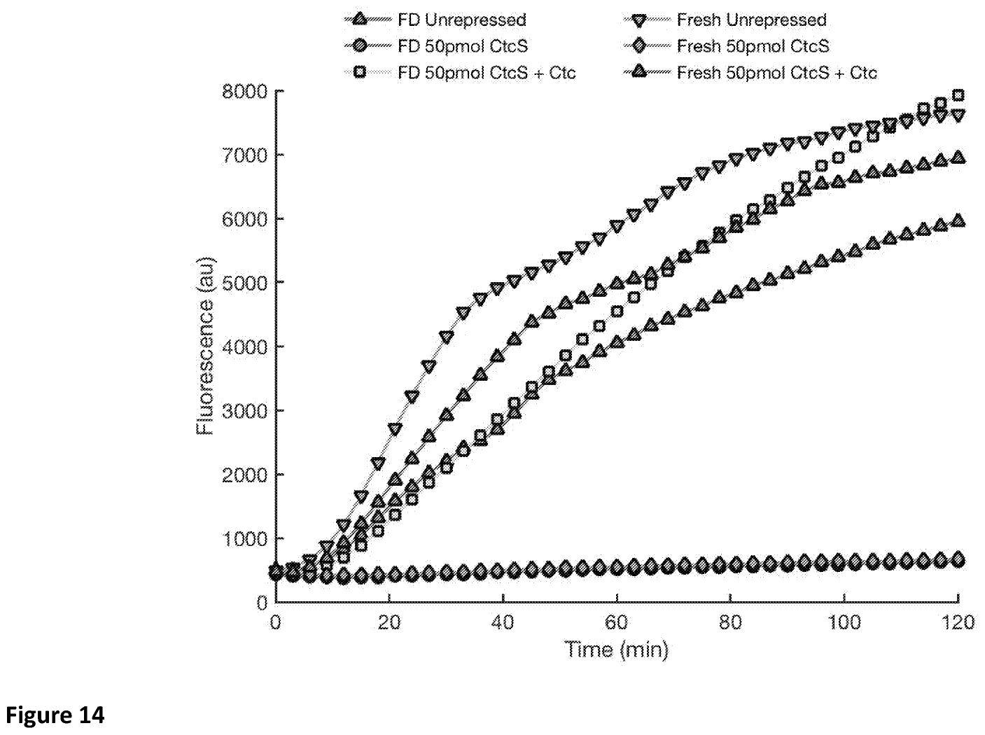

In FIG. 14, we tested the aTF CtcS, which represses transcription unless induced by chlortetracycline (Ctc). Both the fresh and freeze-dried reaction show similar behavior in which the sensor produces signal only in the absence of repressor (“unrepressed”) or with induction of Ctc. (See FIG. 14).

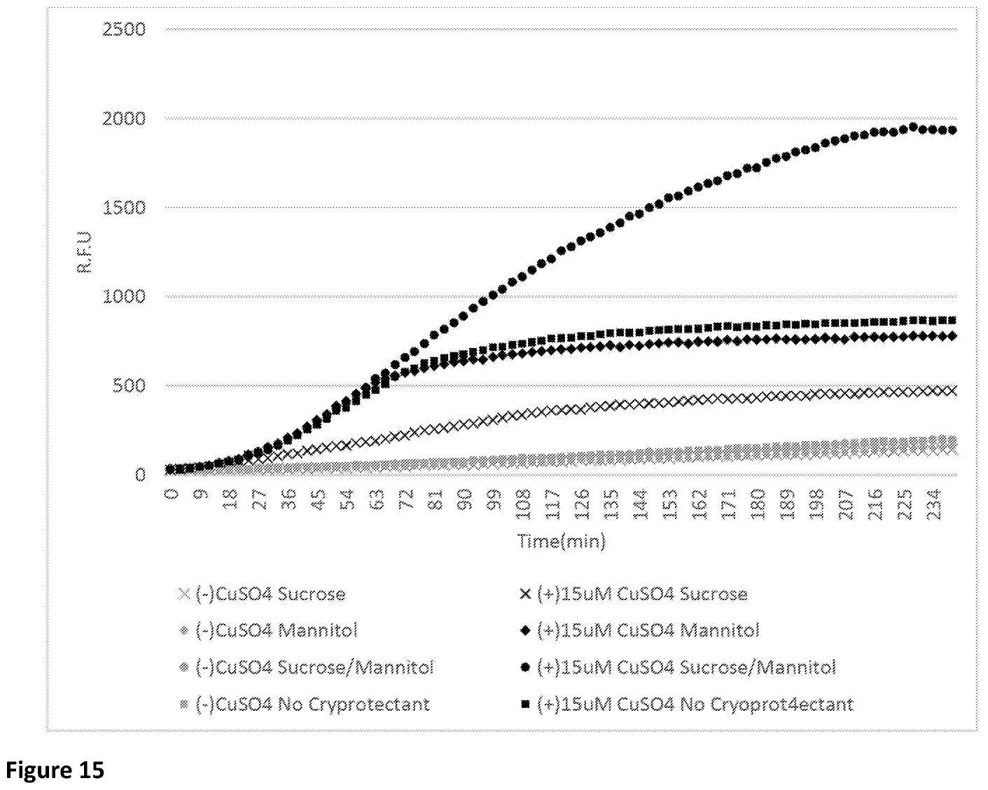

We next tested whether the combination of sucrose and mannitol protected a regulated IVT using the copper sensor, CsoR, and showed induction in the presence of 15 μM CuSO₄. (See FIG. 15).

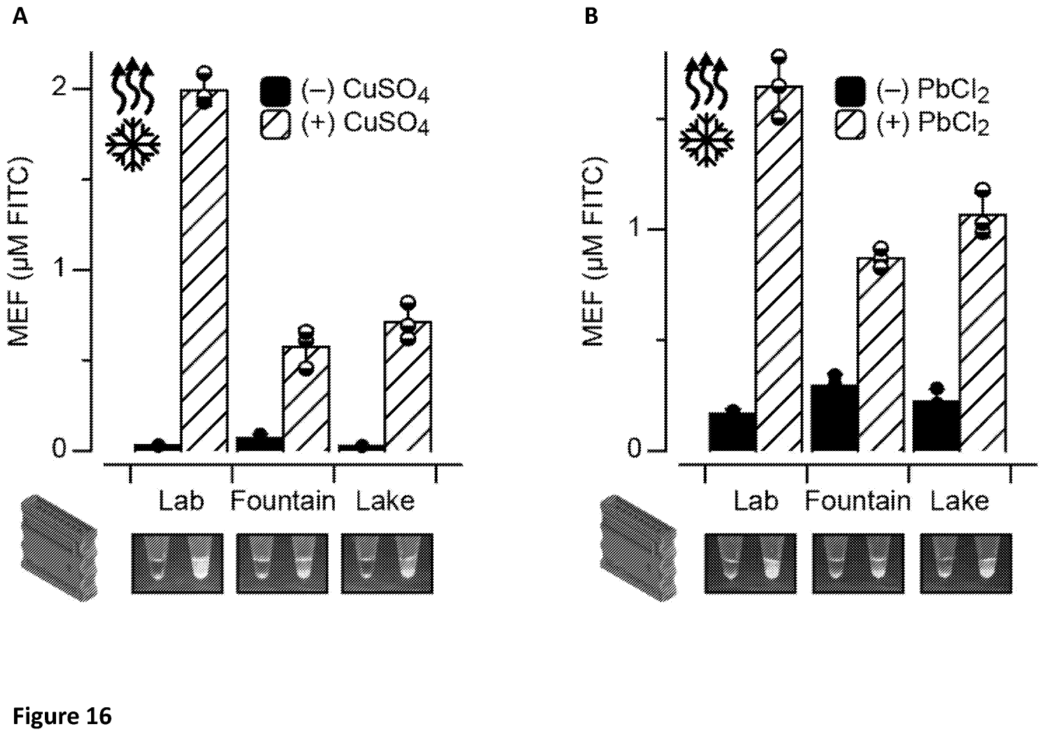

When lyophilized with 5% (50 mM) sucrose and 25% (250 mM) mannitol, we show that the copper sensor (using CsoR as the repressor) and lead sensor (using CadC) remained functional when rehydrated with real world water samples. (See FIG. 16).

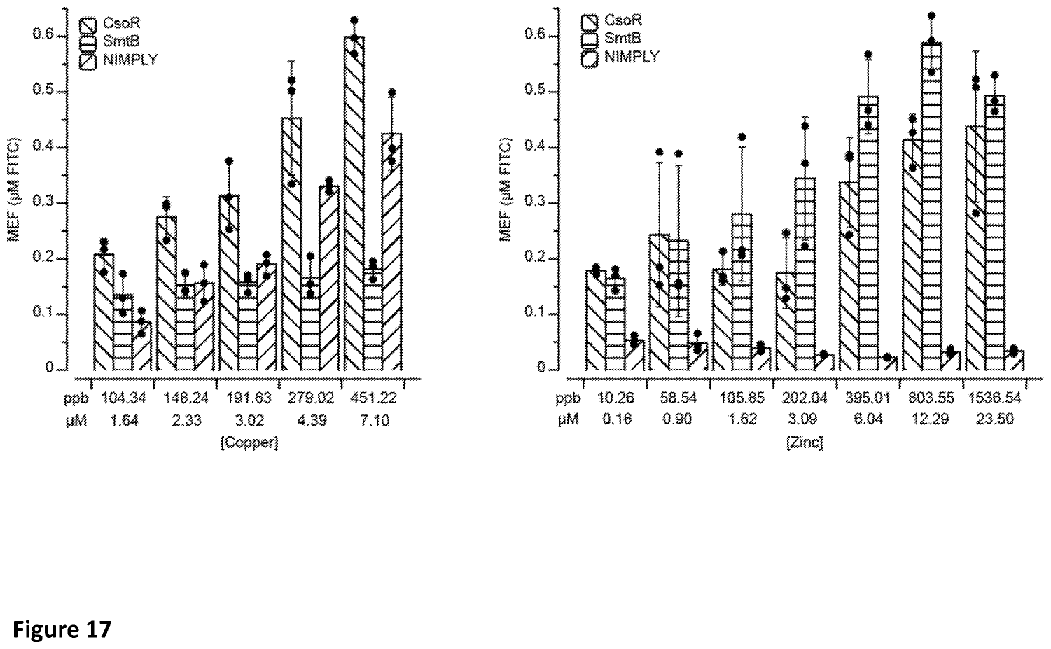

We next applied various real-world water sources such as lake and tap water to our lyophilized regulated IVT reactions. When applied to a tap water source spiked with different concentrations of copper or zinc, our lyophilized copper sensors, zinc sensors (using SmtB as the repressor), and copper and not zinc sensor built using a NIMPLY logic gate (using CsoR and SmtB as the repressors) remained functional, activating in a dose-response manner. (See FIG. 17). Note that our copper sensor has a known promiscuity and responds to zinc as well. This feature is mitigated by our NIMPLY sensor which activates in the presence of copper only.

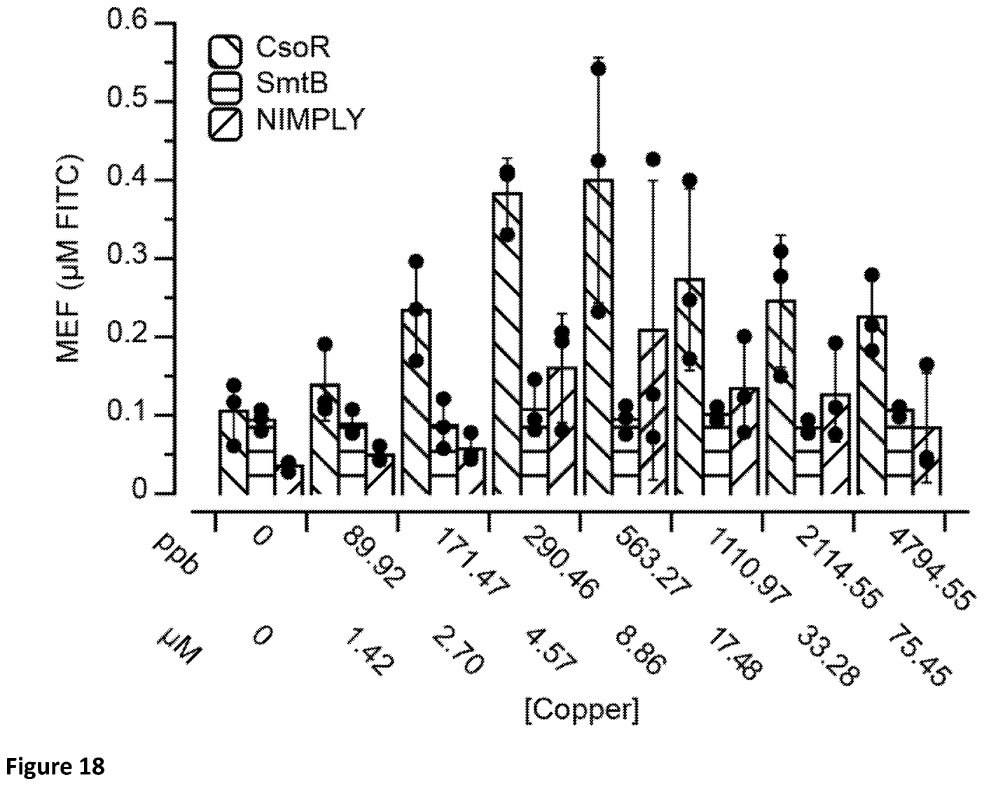

Next, we applied our lyophilized copper sensor, zinc sensor, and NIMPLY sensor to an environmental water source such as lake water spiked with different concentrations of copper. (See FIG. 18). Similar to the tap water results, we observed that these sensors remained functional upon rehydration, again activating in a dose-response manner.

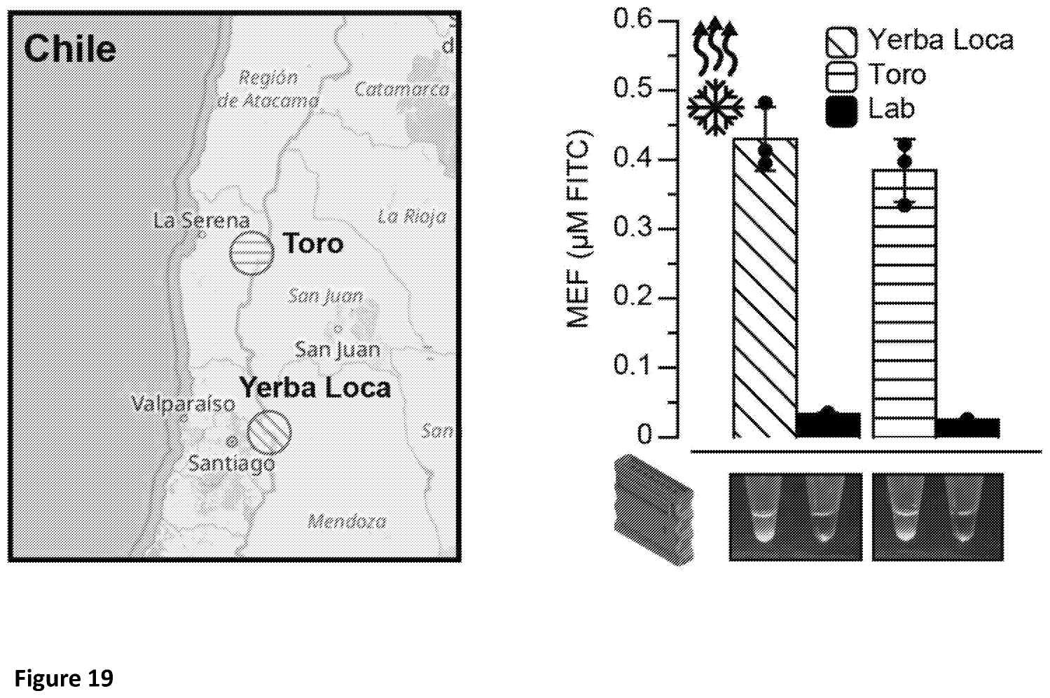

Finally, we applied our lyophilized copper sensors to an environment water source shipped from Chile with known copper contamination, our lyophilized copper sensor remained functional. (See FIG. 19). Our lyophilized copper sensors activated only when exposed to the contaminated water source and not to laboratory water (without an activating amount of copper).

Packaging and Storage of Lyophilized IVT Reactions

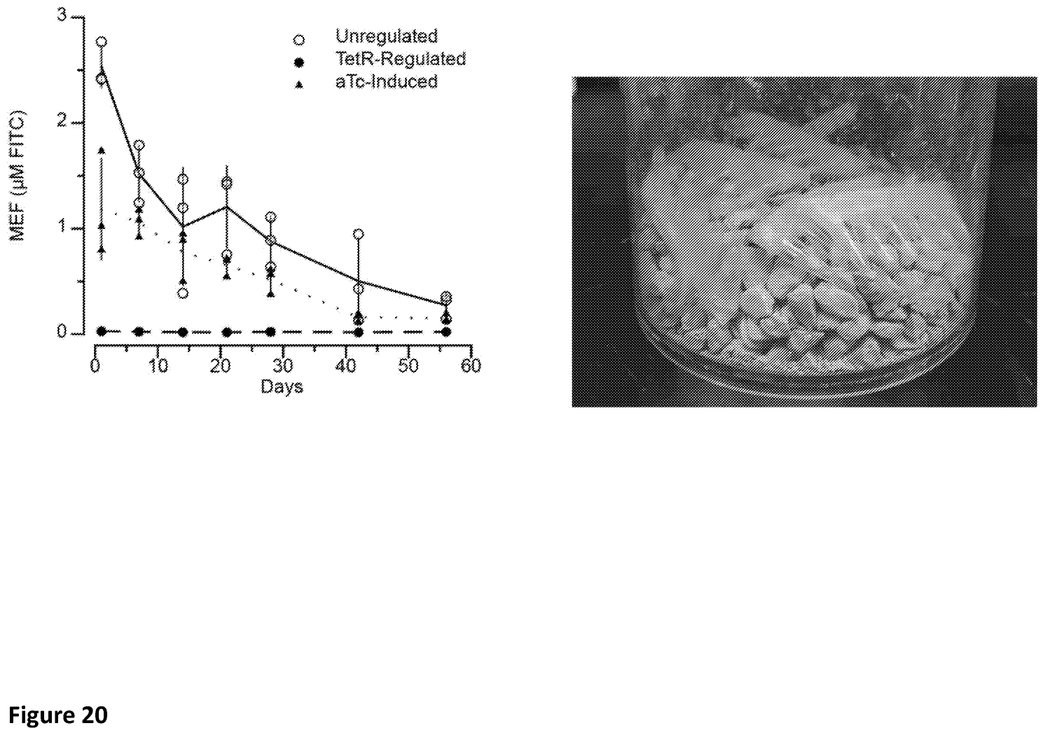

We observed that the packaging process and storage method of lyophilized IVT reactions greatly impact the shelf-life of these reactions. When the tubes with lyophilized reactions were parafilmed and stored in a container filled with drierite desiccants with no light-protection, we observed rapid decrease in activity over time for both unregulated and regulated IVT reactions where TetR was used to regulate and aTC was used to induce the reactions. (See FIG. 20).

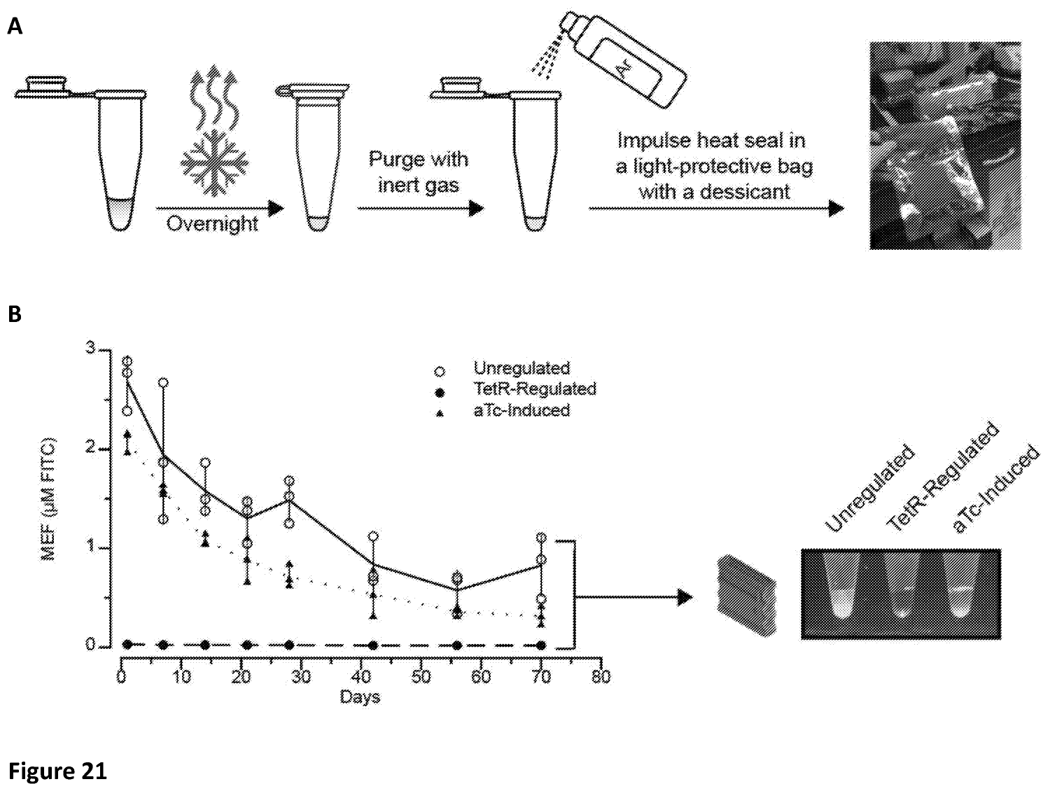

We modified the packaging process and storage method to increase the shelf-life as follows: we first placed the tubes containing lyophilized reactions and a dri-card desiccant in a light-protective mylar bag and purged them with inert gas, specifically argon. Immediately thereafter, we impulse heat-sealed the bag and stored it in a cool, shaded area before rehydration. (See FIG. 21A). When this modified process was applied, we observed a noticeable improvement with an increase in shelf-life up to 2.5 months. (See FIG. 21B).

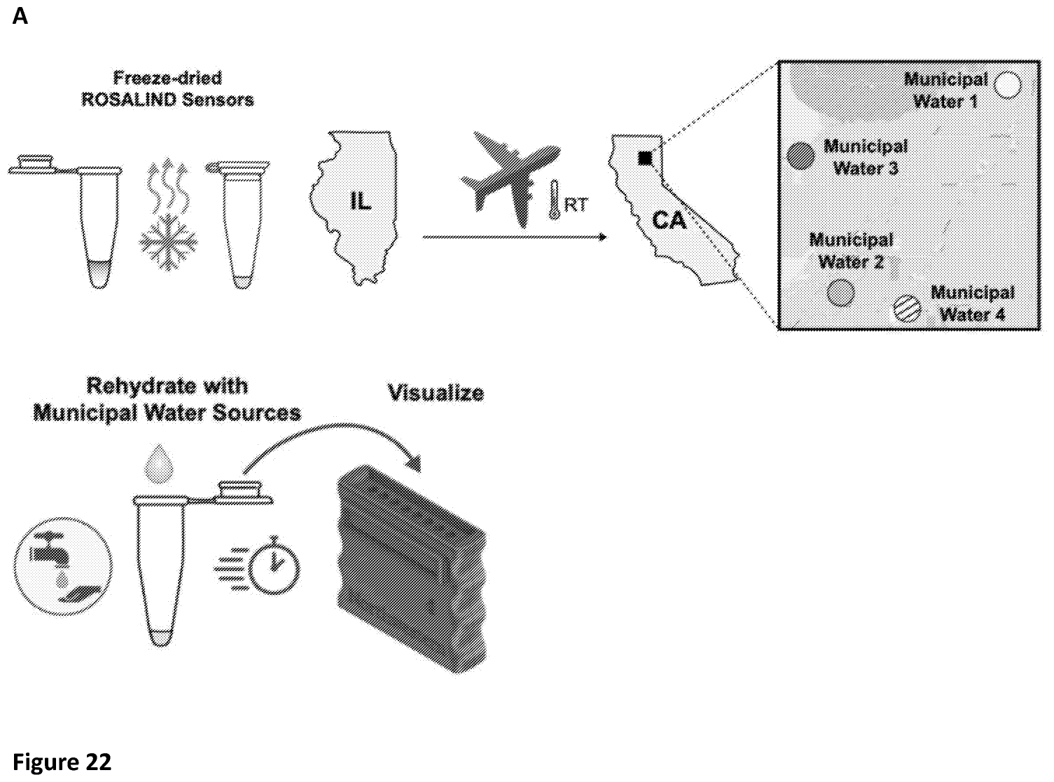

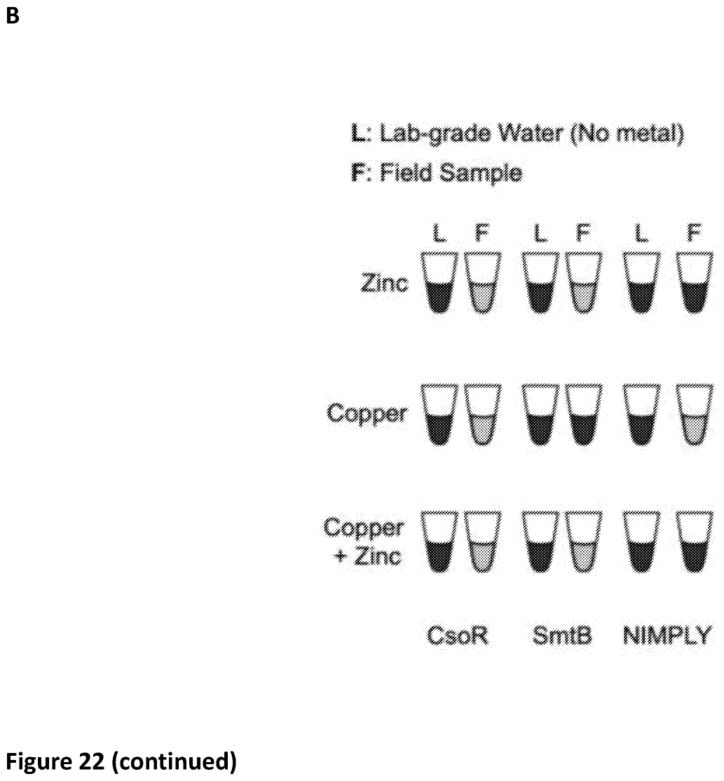

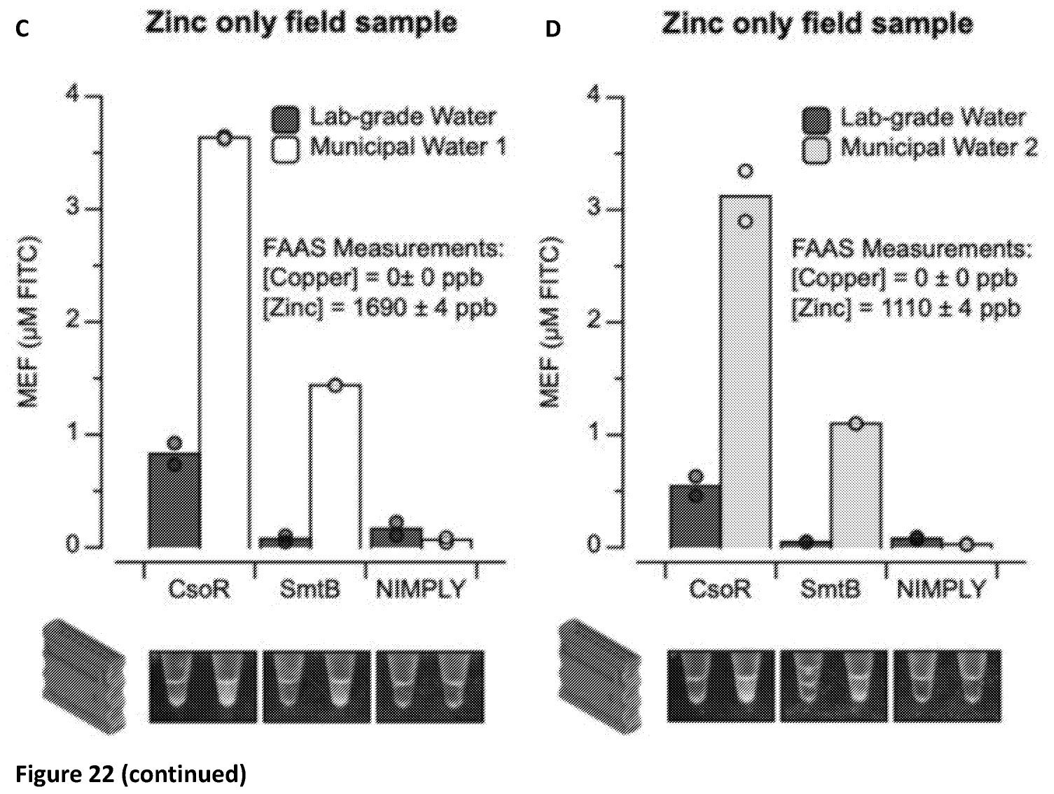

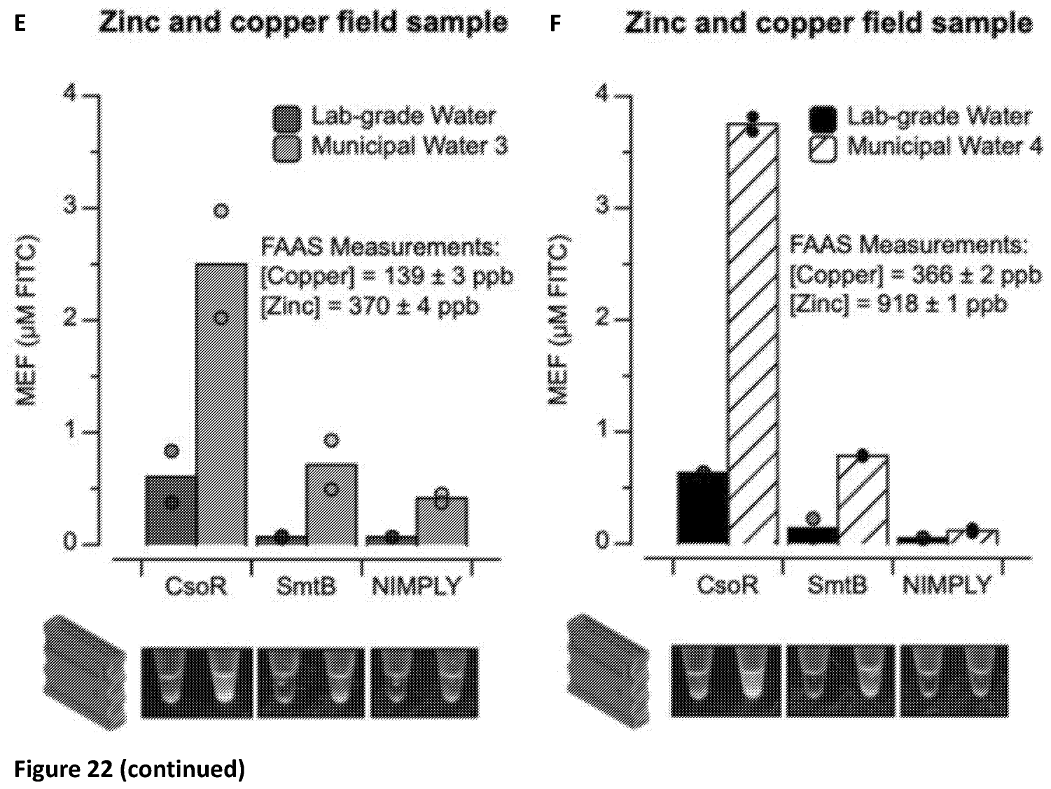

Finally, we found that lyophilized IVT reactions can be packaged, shipped to a location with municipal water samples of interests, and rehydrated on site. (See FIG. 22A). Tap water sources contaminated with zinc and/or copper were used to rehydrate these sensors on site, and we observed expected signals. (See FIGS. 22B, C, and D).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A lyophilized composition comprising components for performing transcription in vitro prepared by lyophilizing an aqueous composition comprising: (i) the components for performing transcription in vitro; and (ii) a non-reducing polysaccharide at a concentration of at least about 40 mM and/or a sugar alcohol at a concentration of at least about 40 mM.

2. The lyophilized composition of claim 1, wherein the non-reducing polysaccharide is a non-reducing disaccharide.

3. The lyophilized composition of claim 1, wherein the non-reducing polysaccharide is selected from sucrose, trehalose, maltotriose, raffinose, or a mixture thereof.

4. The lyophilized composition of claim 1, wherein the non-reducing polysaccharide is present in the aqueous composition at a concentration of at least about 40 mM.

5. The lyophilized composition of claim 1 wherein the sugar alcohol is a 6-carbon sugar alcohol, a 5-carbon sugar alcohol, or a mixture thereof.

6. The lyophilized composition of claim 1, wherein the sugar alcohol is selected from D-mannitol, sorbitol, xylitol, or a mixture thereof.

7. The lyophilized composition of claim 1, wherein the sugar alcohol is present in the aqueous composition at a concentration of at least about 40 mM.

8. The lyophilized composition of claim 1, wherein the aqueous composition comprises no more than 5% (v/v) glycerol.

9. The lyophilized composition of claim 1, wherein the aqueous composition comprises no more than 5% (v/v) organic solvent.

10. The lyophilized composition of claim 1, wherein the aqueous composition comprises one or more components for performing transcription in vitro selected from (i) an RNA polymerase; (ii) a DNA transcription template; (iii) nucleotide triphosphates; and/or (iv) a buffering agent.

11. The lyophilized composition of claim 10, wherein the aqueous composition further comprises one or more components selected from a salt, a metal ion, a reducing agent, a polyamine, an RNase inhibitor, inorganic pyrophosphatase, bovine serum albumin, and/or purified transcription factors.

12. The lyophilized composition of claim 10, wherein the RNA polymerase is selected from E. coli RNA polymerase, T7 RNA polymerase, T3 RNA polymerase, and SP6 RNA polymerase.

13. The lyophilized composition of claim 10, wherein the DNA transcription template encodes an aptamer.

14. The lyophilized composition of claim 13, wherein the aqueous composition further comprises a dye that fluoresces in the presence of the aptamer.

15. The lyophilized composition of claim 1, wherein after water is added to the lyophilized composition to prepare a rehydrated composition, RNA transcription occurs in the rehydrated composition.

16. A method for detecting an analyte in an aqueous sample, the method comprising: (i) adding the aqueous sample to the lyophilized composition of claim 1 to prepare a rehydrated composition, wherein RNA transcription occurs in the rehydrated composition if the analyte is present in the aqueous sample; and (ii) detecting RNA transcription in the rehydrated composition.

17. The method of claim 16, wherein the rehydrated composition comprises a dye that fluoresces in the presence of an aptamer and RNA transcription synthesizes the aptamer.

18. A method for preparing the lyophilized composition of claim 1, the method comprising lyophilizing an aqueous composition comprising: (i) components for performing transcription in vitro; and (ii) a non-reducing polysaccharide at a concentration of at least about 40 mM and/or a sugar alcohol at a concentration of at least about 40 mM.

19. The method of claim 18, wherein the non-reducing polysaccharide is selected from sucrose, trehalose, maltoriose, raffinose, or a mixture thereof, and the non-reducing polysaccharide is present in the aqueous composition at a concentration of at least about 40.

20. The method of claim 18, wherein the sugar alcohol is selected from D-mannitol, sorbitol, xylitol, or a mixture thereof and the sugar alcohol is present in the aqueous composition at a concentration of at least about 40 mM.

21. The method of claims 18, wherein the aqueous composition comprises no more than 5% (v/v) glycerol.

22. The method of claim 18, wherein the aqueous composition comprises no more than 5% (v/v) organic solvent.
